(19) United States
(12) Patent Application Publication
(10) Pub. No.: US 2005/0112650 A1
(43) Pub. Date: May 26, 2005

(54) Reactive polyurethane-based polymers

(75) Inventors: Daniel Chang, Danville, CA (US); Scot Weinberger, Montara, CA (US)

Correspondence Address: MORGAN, LEWIS & BOCKIUS LLP (SF), 2 PALO ALTO SQUARE, PALO ALTO, CA, 94306, US

(73) Assignee: Ciphergen Biosystems, Inc., Fremont, CA (US)

(21) Appl. No.: 10/965,092

(22) Filed: Oct. 14, 2004

Related U.S. Application Data
(60) Provisional application No. 60/513,000, filed on Oct. 20, 2003.

(51) Int. Cl. C12Q 1/68; C12M 1/34

(52) U.S. Cl. 435/6; 435/287.2

(57) ABSTRACT

Polyurethane polymers bearing multiple reactive groups are readily prepared from easily accessible precursors. The reactive groups of the polymers are then derivatized with binding functionalities for analytes, energy absorbing molecules for matrix assisted laser desorption/ionization mass spectrometry, fluorescent moieties and the like. The reactive groups can also be converted to different reactive groups having a desired avidity or specificity for a selected reaction partner. The polymers are incorporated into devices of use for the analysis, capture, separation, or purification of an analyte. In an exemplary embodiment, the invention provides a substrate coated with a polymer of the invention, the substrate being adapted for use as a probe for a mass spectrometer.

This application claims priority to U.S. Provisional Patent Application No. 60/513,000, filed on Oct. 20, 2003, the disclosure of which is incorporated herein by reference in its entirety for all purposes.

### BACKGROUND OF THE INVENTION

Bioassays are used to probe for the presence and/or the quantity of an analyte material in a biological sample. In surface based assays, the analyte species captured and detected on a solid support. An example of a surface-based assay is a DNA microarray. The use of DNA microarrays has become widely adopted in the study of gene expression and genotyping due to the ability to monitor large numbers of genes simultaneously (Schena et al., Science 270:467-470 (1995); Pollack et al., Nat. Genet. 23:41-46 (1999)). Arrays can also be fabricated using other binding moieties such as antibodies, proteins, haptens or aptamers, in order to facilitate a wide variety of bioassays in array format.

Laser desorption mass spectrometry is a particularly useful tool for detecting proteins. SELDI is a method of laser desorption mass spectrometry in which the surface of a mass spectrometry probe plays an active part in the analytical process, either through capture of the analytes through selective adsorption onto the surface (“affinity mass spectrometry”), or through assisting desorption and ionization through attachment of energy absorbing molecules to the probe surface (“surface-enhanced neat desorption” or “SEND”). These methods are described in the art. See, for example, U.S. Pat. Nos. 5,719,060 and 6,225,047, both to Hutchens and Yip.

Probes with functionalized surfaces for SELDI also are known in the art. International publication WO 00/66265 (Rich et al., “Probes for a Gas Phase Ion Spectrometer,” Nov. 9, 2000) describes probes have surfaces with a hydrogel attached functionalized for adsorption of analytes. U.S. patent application U.S. 2003 0032043 A1 (Pohl and Papanu, “Latex Based Adsorbent Chip,” Jul. 16, 2002) describes a probe whose surfaces comprises functionalized latex particles. See, U.S. Pat. Nos. 5,877,297; 5,594,151; 4,979,959; 5,002,582; 5,258,041; 5,512,329; 5,741,551 and 4,839,278.

An effective functionalized material for bioassay applications must have adequate capacity to immobilize a sufficient amount of an analyte from relevant samples in order to provide a suitable signal when subjected to detection (e.g., mass spectroscopy analysis). Suitable functionalized materials must also provide a highly reproducible surface in order to be gainfully applied to profiling experiments, particularly in assay formats in which the sample and the control must be analyzed on separate adsorbent surfaces, e.g. adjacent chip surfaces.

For example, chips that are not based on a highly reproducible surface chemistry result in significant errors when undertaking assays (e.g., profiling comparisons). The need in the art for new functionalized materials, devices incorporating the materials and methods of forming such materials is illustrated by reference to devices that include a hydrogel component. In general devices that include a hydrogel are formed by the in situ polymerization of the hydrogel on a substrate, e.g., bead, particle, plate, etc. The selectivity and reproducibility of devices that include hydrogels is frequently highly dependent upon a number of experimental variables including, monomer concentration, monomer ratios, initiator concentration, solvent evaporation rate, ambient humidity (in the case when the solvent is water), crosslinker concentration, laboratory temperature, pipetting time, sparging conditions, reaction temperature (in the case of thermal polymerizations), reaction humidity, uniformity of ultraviolet radiation (in the case of UV photopolymerization) and ambient oxygen conditions. While many of these parameters can be controlled in a manufacturing setting, is difficult if not impossible to control all of these parameters impinging upon reproducibility. As a result, in situ polymerization results in relatively poor reproducibility of all parameters from spot-to-spot, chip-to-chip and lot-to-lot.

Thus, there is a need for functionalized materials and devices including these materials that provide reproducible results from assay to assay, are easy to use, and provide quantitative data in multi-analyte systems. Moreover, to become widely accepted, the materials should be inexpensive and simple to make, exhibit low non-specific binding, and be able to be formed into a variety of functional device formats. The availability of a device incorporating a material having the above-described characteristics would significantly affect research, individual point of care situations (doctor's office, emergency room, out in the field, etc.), and high throughput testing applications. The present invention provides functionalized materials having these and other desirable characteristics.

### BRIEF SUMMARY OF THE INVENTION

This invention provides a polyurethane that is usefully polymerized into a hydrogel. The polyurethane of this invention is copolymer between at least two species that include a reactive functionalities that combine to form a urethane. The polymers of the invention also optionally include an analyte binding functionality, an energy-absorbing matrix molecule (EAM) or a combination thereof.

In an exemplary aspect, the invention provides a polyurethane that is a copolymer formed between: (i) a cross-linking group that includes at least three reactive moieties, e.g., a hydroxyl moiety, a thiol moiety or a combination thereof; (ii) a first monomer that includes two or more reactive moieties, e.g., a hydroxyl moiety, a thiol moiety or a combination thereof; and (iii) a second monomer that includes at least two reactive moieties selected from the group consisting of an isocyanate moiety, an isothiocyanate moiety or a combination thereof. In certain embodiments, the polyurethane also has incorporated a moiety derived from a polymerizable energy absorbing matrix molecule (EAM), an analyte binding functionality or a combination thereof.

In another aspect, this invention provides a polyurethane-based hydrogel. The hydrogel includes an analyte binding functionality, an energy absorbing moiety or a combination thereof, and cross-linked polyurethane moieties. The polyurethane moieties are a product of a reaction between polyurethane units, each unit comprising a plurality of isocyanate or isothiocyanate moieties and a plurality of internal urethane bonds. Links between the units are formed from the reaction of isocyanate or isothiocyante moieties with internal urethane bonds. In one embodiment, the polyurethane units are those units described above.

The invention also provides a device that incorporates a polyurethane hydrogel of the invention. An exemplary device includes a solid support having a surface. The polyurethane hydrogel is immobilized on the surface.

An exemplary device of the invention includes a substrate and a functionalized film, formed from a polyurethane of the invention, which is attached covalently to the substrate. The nature of the substrate depends upon the intended application of the functionalized material. In a preferred embodiment the substrate can also be in the form of a plate or a chip. In an exemplary embodiment, the device is a chip for use in conjunction with mass spectrometry, e.g., the substrate is configured to engage. If the chip is to be used in linear time-of-flight mass spectrometry, the substrate preferably includes a conductive material, such as a metal. If the biochip is to be used in mass spectrometry involving orthogonal extraction, the substrate preferably includes a non-conductive material. If the biochip is to be used in another detection method, such as fluorescence detection at the biochip surface, suitable materials, such as plastics or glass can be used.

Alternatively, if the material is to be utilized for chromatographic separation, such as affinity chromatography, the substrate can be formed from a suitable chromatographic material that is suitably configured. Thus, the substrate is optionally in the form of beads or particles.

The substrate typically will have functional groups through which the hydrogel is immobilized. For example, an aluminum chip contains surface Al—OH groups. Also, it can be coated with silicon dioxide. Other metals, such as anodized aluminum have surfaces with functional groups. Alternatively, the substrate may be composed of plastic in which case the functional groups may already be present as an integral surface component or the surface may be derivatized, making use of methods well-known to those skilled in the art. The devices of the invention may also include a linker arm between the substrate and the functionalized material, serving to anchor the functionalized material to the substrate.

The hydrogel of the invention is highly versatile, allowing the incorporation of a wide variety of binding functionalities. In certain embodiments, the functionalities can be positively charged (anion exchange), negatively charged (cation exchange), a chelating agent, e.g., that can engage in coordinate covalent bonding with a metal ion or a biospecific compound, e.g., an antibody or cellular receptor. Preferred compounds for derivatization include N,N,N-trimethylethanolammonium salt (e.g., chloride) N,N-dimethylethanolamine (strong anion exchange or “SAX”), N,N-dimethyloctylamine (SAX), N-methylglucamine (weak anion exchange or “WAX”), 3-mercaptopropane sulfonate (strong cation exchange or “SCX”), 3-mercaptopropionate, dimethyloacetic acid, dihydroxybenzoic acid, (weak cation exchange or “WCX”) or N,N-bis(carboxymethyl)-L-lysine or N-hydroxyethylethylenediaminoe-triacetic acid (NTA) (immobilized metal chelate or “IMAC”).

In another aspect, this invention provides a method for detecting an analyte in a sample. The method includes contacting the analyte with an adsorbent polyurethane of the invention to allow capture of the analyte and detecting capture of the analyte by the functionalized material. In certain embodiments, the analyte is a biomolecule, such as a polypeptide, a polynucleotide, a carbohydrate, a lipid, or hybrids thereof. In other embodiments, the analyte is an organic molecule such as a drug, drug candidate, cofactor or metabolite. In another embodiment, the analyte could be an inorganic molecule, such as a metal complex or cofactor.

Detection of the analyte can be accomplished by any art-recognized method or device. In certain embodiments, the analyte is detected by mass spectrometry, in particular by laser desorption/ionization mass spectrometry. In such methods, when the analyte is a biomolecule, the method preferably comprises applying a matrix to the captured analyte before detection. Alternatively, a component of an energy absorbing matrix is copolymerized into the structure of the functionalized material. In other embodiments the analyte is labeled, e.g., fluorescently, and is detected on the device by a detector of the label, e.g., a fluorescence detector such as a CCD array. In certain embodiments the method involves profiling a certain class of analytes (e.g., biomolecules) in a sample by applying the sample to one or addressable locations of the device and detecting analytes captured at the addressable location or locations.

### BRIEF DESCRIPTION OF THE DRAWINGS

FIG. 1 shows an exemplary polyurethane polymer unit (T-gel) of this invention, including the constituent monomer units: cross-linking monomer, first monomer, second monomer and functional monomer.

FIG. 2 shows the polymerization reaction involving two T-gel units which results in the formation of a hydrogel. An isocyanate moiety from one T-gel unit reacts with a urethane bond of another T-gel unit. The reactions are similar if the reactive groups comprise isothiocyanates and sulfur-containing urethane bonds.

FIG. 3 is a display of exemplary reaction pathways available for functionalizing activated polyurethane (T-Gel) of the invention with reactive functionalities.

FIG. 4 is a display of exemplary reaction pathways available for preparing a polymer having a biospecific binding functionality, which is based upon the polyurethane (T-Gel) of the invention. These biochips are created by coupling a biospecific moiety to a reactive hydrogel such as shown in FIG. 3.

FIG. 5 is a display of exemplary reaction pathways available for preparing a chromatographic polymer based upon the polyurethane of the invention.

FIG. 6 is a display of exemplary reaction pathways available for preparing SEND (Surface Enhanced for Neat Desorption) chips of the invention having energy absorbing moieties.

FIG. 7 is a display of exemplary reaction pathways available for preparing a chromatographic polymer SEND (Surface Enhanced for Neat Desorption) chips of the invention having energy absorbing moieties and based upon the polyurethane of the invention FIG. 8 is a display of exemplary reaction pathways available for preparing hydrogels comprising both EAM (SEND) and activated binding functionalities.

FIG. 9 shows a laser desorption/ionization mass spectrum of a seven-peptide mixture applied to a CHCA-PU 200 SEND chip from Example 4.1a. The seven-peptide mixture includes Arg-vasopressin (MW 1084.2), Somatostatin (MW 1637.9), Dynorphin (MW 2147.5), ACTH (Human) (MW 2833.5), Bovine Insulin B-Chain (MW 3495.9), Human Insulin (MW 5807.7) and Hirudin BHVK (MW 7033.6). The peptides are suspended in 50 ul of Buffer (10 mM ammonium acetate, 25% acetonitrile, 1.25% of trifluoroacetic acid). 1 ul of the solution was spotted on the PU SEND chip and allow it to dry. The peptides were detected on a Ciphergen PBS II mass spectrometer to obtain the spectra.

FIG. 10 is an exemplary solid support capable of engaging a probe of a mass spectrometer.

### DETAILED DESCRIPTION OF THE INVENTION

I. Abbreviations

NHS(N-hydroxysuccinimide); PDS (pyridinyl disulfide); PNP (para-nitrophenylcarbonate); NHM (N-hydroxymaleimide); PFP (Parafluorophenol); EAM (energy absorbing moiety); PVA (Polyvinyl alcohol) NTA,(N-hydroxyethylethylenediaminoe-triacetic acid), SPA (Sinapinic acid), CHCA (alpha-cyano-4-hydroxy-succininc acid), TMP (trimethylol propane), PNP (p-nitrophenol).

II. Definitions

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and nucleic acid chemistry and hybridization described below are those well known and commonly employed in the art. Standard techniques are used for nucleic acid and peptide synthesis. The techniques and procedures are generally performed according to conventional methods in the art and various general references, which are provided throughout this document. The nomenclature used herein and the laboratory procedures in analytical chemistry, and organic synthetic described below are those well known and commonly employed in the art. Standard techniques, or modifications thereof, are used for chemical syntheses and chemical analyses.

The terms “host” and “molecular host” refer, essentially interchangeably, to a molecule that surrounds or partially surrounds and attractively interacts with a molecular “guest.” When the “host” and “guest” interact the resulting species is referred to herein as a “complex.”

Where substituent groups are specified by their conventional chemical formulae, written from left to right, they equally encompass the chemically identical substituents which would result from writing the structure from right to left, e.g., —CH₂O— is intended to also recite —OCH₂—; —NHS(O)₂— is also intended to represent. —S(O)₂HN—, etc.

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below, such as “heteroalkyl.” Alkyl groups, which are limited to hydrocarbon groups are termed “homoalkyl”.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si and S, and wherein the nitrogen and sulfur atoms may optionally be oxidized and the nitrogen heteroatom may optionally be quaternized. The heteroatom(s) O, N and S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′-represents both —C(O)₂R′- and —R° C(O)₂—.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, ═N—OR′, —NR′R″, —SR′, -halogen, —SiR′R″ R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′)═NR“ ”, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R“ ” each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Each of the above terms is meant to include both substituted and unsubstituted forms of the indicated radical.

As used herein, the term “heteroatom” is meant to include oxygen (O), nitrogen (N), sulfur (S) and silicon (Si).

“Binding functionality,” or “analyte binding functionality,” as used herein means a moiety, which has an affinity for a certain substance such as a “substance to be assayed,” that is, a moiety capable of interacting with a specific substance to immobilize it on an adsorbent material of the invention. Binding functionalities can be chromatographic or biospecific. Chromatographic binding functionalities bind substances via charge-charge, hydrophilic-hydrophilic, hydrophobic-hydrophobic, van der Waals interactions and combinations thereof. Biospecific binding functionalities generally involve complementary 3-dimensional structures involving one or more of the above interactions. Examples of combinations of biospecific interactions include, but are not limited to, antigens with corresponding antibody molecules, a nucleic acid sequence with its complementary sequence, effector molecules with receptor molecules, enzymes with inhibitors, sugar chain-containing compounds with lectins, an antibody molecule with another antibody molecule specific for the former antibody, receptor molecules with corresponding antibody molecules and the like combinations. Other examples of the specific binding substances include a chemically biotin-modified antibody molecule or polynucleotide with avidin, an avidin-bound antibody molecule with biotin and the like combinations.

“Molecular binding partners” and “specific binding partners” refer to pairs of molecules, typically pairs of biomolecules that exhibit specific binding. Molecular binding partners include, without limitation, receptor and ligand, antibody and antigen, biotin and avidin, and biotin and streptavidin.

“Adsorbent film” as used herein means an area where a substance to be assayed is immobilized and a specific binding reaction occurs. The reaction optionally has a distribution along the flow direction of a test sample.

As used herein, the terms “polymer” and “polymers” include “copolymer” and “copolymers,” and are used interchangeably with the terms “oligomer” and “oligomers.”

“Attached,” as used herein encompasses interaction including chemisorption and physisorption, e.g., covalent bonding, ionic bonding, and combinations thereof.

“Independently selected” is used herein to indicate that the groups so described can be identical or different.

“Analyte” refers to any component of a sample that is desired to be detected. The term can refer to a single component or a plurality of components in the sample. Analytes include, for example, biomolecules. Biomolecules can be sourced from any biological material.

“Biomolecule” or “bioorganic molecule” refers to an organic molecule typically made by living organisms. This includes, for example, molecules comprising nucleotides, amino acids, sugars, fatty acids, steroids, nucleic acids, polypeptides, peptides, peptide fragments, carbohydrates, lipids, and combinations of these (e.g., glycoproteins, ribonucleoproteins, lipoproteins, or the like).

“Biological material” refers to any material derived from an organism, organ, tissue, cell or virus. This includes biological fluids such as saliva, blood, urine, lymphatic fluid, prostatic or seminal fluid, milk, etc., as well as extracts of any of these, e.g., cell extracts or lysates (from, e.g., primary tissue or cells, cultured tissue or cells, normal tissue or cells, diseased tissue or cells, benign tissue or cells, cancerous tissue or cells, salivary glandular tissue or cells, intestinal tissue or cells, neural tissue or cells, renal tissue or cells, lymphatic tissue or cells, bladder tissue or cells, prostatic tissue or cells, urogenital tissues or cells, tumoral tissue or cells, tumoral neovasculature tissue or cells, or the like), cell culture media, fractionated samples (e.g., serum or plasma), or the like. For example, cell lysate samples are optionally derived.

“Gas phase ion spectrometer” refers to an apparatus that detects gas phase ions. Gas phase ion spectrometers include an ion source that supplies gas phase ions. Gas phase ion spectrometers include, for example, mass spectrometers, ion mobility spectrometers, and total ion current measuring devices. “Gas phase ion spectrometry” refers to the use of a gas phase ion spectrometer to detect gas phase ions.

“Mass spectrometer” refers to a gas phase ion spectrometer that measures a parameter that can be translated into mass-to-charge ratios of gas phase ions. Mass spectrometers generally include an ion source and a mass analyzer. Examples of mass spectrometers are time-of-flight, magnetic sector, quadrupole filter, ion trap, ion cyclotron resonance, electrostatic sector analyzer and hybrids of these. “Mass spectrometry” refers to the use of a mass spectrometer to detect gas phase ions.

“Laser desorption mass spectrometer” refers to a mass spectrometer that uses laser energy as a means to desorb, volatilize, and ionize an analyte.

“Mass analyzer” refers to a sub-assembly of a mass spectrometer that comprises means for measuring a parameter that can be translated into mass-to-charge ratios of gas phase ions. In a time-of-flight mass spectrometer the mass analyzer comprises an ion optic assembly, a flight tube and an ion detector.

“Ion source” refers to a sub-assembly of a gas phase ion spectrometer that provides gas phase ions. In one embodiment, the ion source provides ions through a desorption/ionization process. Such embodiments generally comprise a probe interface that positionally engages a probe in an interrogatable relationship to a source of ionizing energy (e.g., a laser desorption/ionization source) and in concurrent communication at atmospheric or subatmospheric pressure with a detector of a gas phase ion spectrometer.

Forms of ionizing energy for desorbing/ionizing an analyte from a solid phase include, for example: (1) laser energy; (2) fast atoms (used in fast atom bombardment); (3) high energy particles generated via beta decay of radionucleides (used in plasma desorption); and (4) primary ions generating secondary ions (used in secondary ion mass spectrometry). The preferred form of ionizing energy for solid phase analytes is a laser (used in laser desorption/ionization), in particular, nitrogen lasers, Nd-Yag lasers and other pulsed laser sources. “Fluence” refers to the energy delivered per unit area of interrogated image. A high fluence source, such as a laser, will deliver about 1 mJ/mm² to about 50 mJ/mm². Typically, a sample is placed on the surface of a probe, the probe is engaged with the probe interface and the probe surface is exposed to the ionizing energy. The energy desorbs analyte molecules from the surface into the gas phase and ionizes them.

Other forms of ionizing energy for analytes include, for example: (1) electrons that ionize gas phase neutrals; (2) strong electric field to induce ionization from gas phase, solid phase, or liquid phase neutrals; and (3) a source that applies a combination of ionization particles or electric fields with neutral chemicals to induce chemical ionization of solid phase, gas phase, and liquid phase neutrals.

“Surface-enhanced laser desorption/ionization” or “SELDI” refers to a method of desorption/ionization gas phase ion spectrometry (e.g., mass spectrometry) in which the analyte is captured on the surface of a SELDI probe that engages the probe interface of the gas phase ion spectrometer. In “SELDI MS,” the gas phase ion spectrometer is a mass spectrometer. SELDI technology is described in, e.g., U.S. Pat. No. 5,719,060 (Hutchens and Yip) and U.S. Pat. No. 6,225,047 (Hutchens and Yip).

“Surface-Enhanced Affinity Capture” (“SEAC”) or “affinity gas phase ion spectrometry” (e.g., “affinity mass spectrometry”) is a version of the SELDI method that uses a probe comprising an absorbent surface (a “SEAC probe”). “Adsorbent surface” refers to a sample presenting surface of a probe to which an adsorbent (also called a “capture reagent” or an “affinity reagent”) is attached. An adsorbent is any material capable of binding an analyte (e.g., a target polypeptide or nucleic acid). “Chromatographic adsorbent” refers to a material typically used in chromatography. “Biospecific adsorbent” refers an adsorbent comprising a biomolecule, e.g., a nucleic acid molecule (e.g., an aptamer), a polypeptide, a polysaccharide, a lipid, a steroid or a conjugate of these (e.g., a glycoprotein, a lipoprotein, a glycolipid, a nucleic acid (e.g., DNA)-protein conjugate). Further examples of adsorbents for use in SELDI can be found in U.S. Pat. No. 6,225,047 (Hutchens and Yip, “Use of retentate chromatography to generate difference maps,” May 1, 2001).

In some embodiments, a SEAC probe is provided as a pre-activated surface that can be modified to provide an adsorbent of choice. For example, certain probes are provided with a reactive moiety that is capable of binding a biological molecule through a covalent bond. Epoxide and carbodiimidizole are useful reactive moieties to covalently bind biospecific adsorbents such as antibodies or cellular receptors.

In a preferred embodiment affinity mass spectrometry involves applying a liquid sample comprising an analyte to the adsorbent surface of a SELDI probe. Analytes, such as polypeptides, having affinity for the adsorbent bind to the probe surface. Typically, the surface is then washed to remove unbound molecules, and leaving retained molecules. The extent of analyte retention is a function of the stringency of the wash used. An energy absorbing material (e.g., matrix) is then applied to the adsorbent surface. Retained molecules are then detected by laser desorption/ionization mass spectrometry.

SELDI is useful for protein profiling, in which proteins in a sample are detected using one or several different SELDI surfaces. In turn, protein profiling is useful for difference mapping, in which the protein profiles of different samples are compared to detect differences in protein expression between the samples.

“Surface-Enhanced Neat Desorption” or “SEND” is a version of SELDI that involves the use of probes (“SEND probe”) comprising a layer of energy absorbing molecules attached to the probe surface. Attachment can be, for example, by covalent or non-covalent chemical bonds. Unlike traditional MALDI, the analyte in SEND is not required to be trapped within a crystalline matrix of energy absorbing molecules for desorption/ionization.

SEAC/SEND is a version of SELDI in which both a capture reagent and an energy absorbing molecule are attached to the sample presenting surface. SEAC/SEND probes therefore allow the capture of analytes through affinity capture and desorption without the need to apply external matrix. The C18 SEND chip is a version of SEAC/SEND, comprising a C18 moiety which functions as a capture reagent, and a CHCA moiety that functions as an energy absorbing moiety.

“Surface-Enhanced Photolabile Attachment and Release” or “SEPAR” is a version of SELDI that involves the use of probes having moieties attached to the surface that can covalently bind an analyte, and then release the analyte through breaking a photolabile bond in the moiety after exposure to light, e.g., laser light. SEPAR is further described in U.S. Pat. No. 5,719,060.

“Eluant” or “wash solution” refers to an agent, typically a solution, which is used to affect or modify adsorption of an analyte to an adsorbent surface and/or remove unbound materials from the surface. The elution characteristics of an eluant can depend, for example, on pH, ionic strength, hydrophobicity, degree of chaotropism, detergent strength and temperature.

“Monitoring” refers to recording changes in a continuously varying parameter.

Data generation in mass spectrometry begins with the detection of ions by an ion detector. A typical laser desorption mass spectrometer can employ a nitrogen laser at 337.1 nm. A useful pulse width is about 4 nanoseconds. Generally, power output of about 1-25 μJ is used. Ions that strike the detector generate an electric potential that is digitized by a high speed time-array recording device that digitally captures the analog signal. Ciphergen's ProteinChip® system employs an analog-to-digital converter (ADC) to accomplish this. The ADC integrates detector output at regularly spaced time intervals into time-dependent bins. The time intervals typically are one to four nanoseconds long. Furthermore, the time-of-flight spectrum ultimately analyzed typically does not represent the signal from a single pulse of ionizing energy against a sample, but rather the sum of signals from a number of pulses. This reduces noise and increases dynamic range. This time-of-flight data is then subject to data processing. In Ciphergen's ProteinChip® software, data processing typically includes TOF-to-M/Z transformation, baseline subtraction, high frequency noise filtering.

TOF-to-M/Z transformation involves the application of an algorithm that transforms times-of-flight into mass-to-charge ratio (M/Z). In this step, the signals are converted from the time domain to the mass domain. That is, each time-of-flight is converted into mass-to-charge ratio, or M/Z. Calibration can be done internally or externally. In internal calibration, the sample analyzed contains one or more analytes of known M/Z. Signal peaks at times-of-flight representing these massed analytes are assigned the known M/Z. Based on these assigned M/Z ratios, parameters are calculated for a mathematical function that converts times-of-flight to M/Z. In external calibration, a function that converts times-of-flight to M/Z, such as one created by prior internal calibration, is applied to a time-of-flight spectrum without the use of internal calibrants.

Baseline subtraction improves data quantification by eliminating artificial, reproducible instrument offsets that perturb the spectrum. It involves calculating a spectrum baseline using an algorithm that incorporates parameters such as peak width, and then subtracting the baseline from the mass spectrum.

High frequency noise signals are eliminated by the application of a smoothing function. A typical smoothing function applies a moving average function to each time-dependent bin. In an improved version, the moving average filter is a variable width digital filter in which the bandwidth of the filter varies as a function of, e.g., peak bandwidth, generally becoming broader with increased time-of-flight. See, e.g., WO 00/70648, Nov. 23, 2000 (Gavin et al., “Variable Width Digital Filter for Time-of-flight Mass Spectrometry”).

A computer can transform the resulting spectrum into various formats for displaying. In one format, referred to as “spectrum view or retentate map,” a standard spectral view can be displayed, wherein the view depicts the quantity of analyte reaching the detector at each particular molecular weight. In another format, referred to as “peak map,” only the peak height and mass information are retained from the spectrum view, yielding a cleaner image and enabling analytes with nearly identical molecular weights to be more easily seen. In yet another format, referred to as “gel view,” each mass from the peak view can be converted into a grayscale image based on the height of each peak, resulting in an appearance similar to bands on electrophoretic gels. In yet another format, referred to as “3-D overlays,” several spectra can be overlaid to study subtle changes in relative peak heights. In yet another format, referred to as “difference map view,” two or more spectra can be compared, conveniently highlighting unique analytes and analytes that are up- or down-regulated between samples.

Analysis generally involves the identification of peaks in the spectrum that represent signal from an analyte. Peak selection can, of course, be done by eye. However, software is available as part of Ciphergen's ProteinChip® software that can automate the detection of peaks. In general, this software functions by identifying signals having a signal-to-noise ratio above a selected threshold and labeling the mass of the peak at the centroid of the peak signal. In one useful application many spectra are compared to identify identical peaks present in some selected percentage of the mass spectra. One version of this software clusters all peaks appearing in the various spectra within a defined mass range, and assigns a mass (M/Z) to all the peaks that are near the mid-point of the mass (M/Z) cluster.

### III. EMBODIMENTS

Introduction

It has now been discovered that a solution to the shortcomings of prior functionalized materials resides in the synthesis of a functionalized film in a process that is separate from the process by which the functionalized material is incorporated into the device, e.g., attached to the substrate of a chip. By separating the attachment of the functionalized material from the manufacture of the device incorporating the film, the individual processes are more readily controlled. Furthermore, if sufficient functionalized material is synthesized using a material of suitable chemical stability, one can readily synthesize enough material to allow the use of a single lot of stationary phase over the entire product lifecycle of a given device of the invention. Quite surprisingly, in an embodiment of the methods set forth herein, approximately one million chips of the invention can be prepared from less than one liter of functionalized material. Thus, using this present method one can produce chips with minimal variability in selectivity over the entire product lifecycle.

This invention provides a biochip comprising a polyurethane-based hydrogel attached to its surface. Preferably, the hydrogel is further functionalized with one or more groups useful for the capture or detection of biomolecules, in particular, proteins.

In one embodiment, the hydrogel results from a three-step process comprising creation of a “T-gel,” functionalizing the T-gel and curing the T-gel.

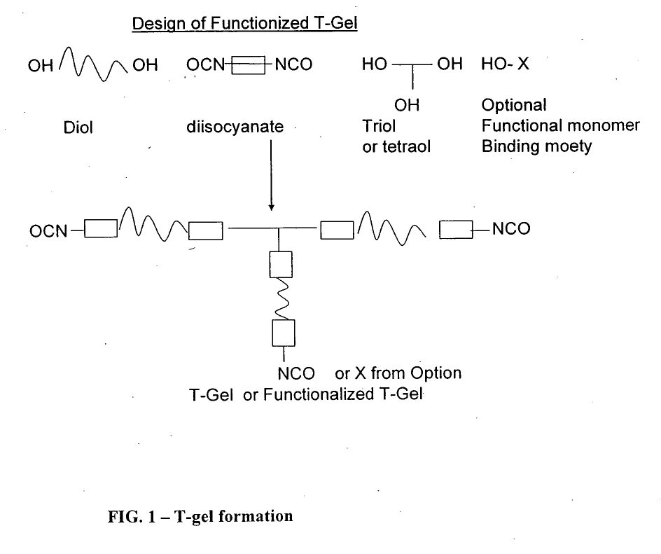

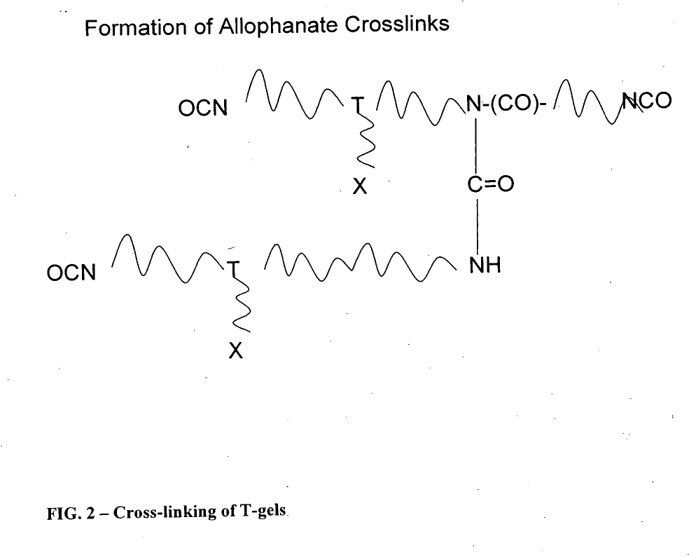

A T-gel of this invention is a polyurethane created by polymerizing three monomers: (1) A triol, tetraol or other polyol, for example trimethylol propane (CH(CH₂OH)₃); (2) a di-isocyanate, for example toluene di-isocyante; and (3) a long-chain diol, such as polyethylene glycol diol (H(—O—CH₂—H₂)ₙ—OH). By controlling the reaction conditions these ingredients can form the T-gel polymer shown in FIG. 1. The isocyanate moieties react with the hydroxyl moieties to form urethane bonds (R—NH—CO—O—R′). See FIG. 2. Polyurethane has many characteristics that are desirable in a functionalized material of the invention. For example, polyurethane exhibits low non-specific binding.

The T-gel is functionalized by reaction with a monomer that includes a group of choice (e.g., binding functionality or EAM) and a group that reacts with an isocyanate, such as a hydroxyl or an amine. In an exemplary embodiment, this reaction is controlled to leave one or more free isocyanate groups on the functionalized T-gel. In one embodiment, the functional moieties on the T-gel function as binding functionalities. For example, the functional moieties can be reactive moieties, such as epoxides, imidazoles, N-hydroxysuccinimide, etc. These groups on the T-gel react to covalently couple to proteins, such as antibodies or receptors, which, in turn, can be used to capture analytes in a sample to which they bind. Also, the functional moieties can be those moieties typically used in chromatography to capture classes of molecules having similar properties, such as hydrophobic or hydrophilic groups, or ion exchange groups or metal chelating groups. Also, the functional moieties can be energy absorbing moieties that facilitate desorption and ionization of analytes in contact with the gel that are addressed by energy from an energy source, for example in laser desorption/ionization mass spectrometry.

The T-gel can be cured to form a cross-linked polyurethane-based polymer that functions as a hydrogel. In particular, a free isocyanate moiety of one T-gel can react with a urethane bond of another T-gel to form a urea bond:
R″—NCO+R—NH—COOR′→R″—NH—CO—NH(R)—CO—O—R′.
Depending on its desired application, the T-gel can be functionalized before curing, or a functionalized monomer can be added to the solution upon or after curing.

In an exemplary embodiment, the T-gel can be cured on the surface of a chip to form a biochip. A biochip comprising the hydrogel of the invention attached to the surface of a solid support will preferably include one or more functional group useful in the capture and/or detection of biomolecules. In one embodiment, the surface comprises free hydroxyl groups (e.g., silicon dioxide, aluminium hydroxide or any metal oxides) or amines (e.g., amino silane) that can react with free isocyante moieties on the T-gel. In this way, the hydrogel can be covalently coupled to the chip surface. Alternatively, the T-gel is cured on an inert surface, in which case the hydrogel becomes physisorbed to the surface.

The Polyurethane Polymer

An exemplary polyurethane polymer of the invention is a copolymer formed between at least a first monomer, a second monomer, a cross-linking monomer and optionally a functional moiety monomer, such as a binding functionality monomer or an EAM monomer. Polyurethanes are based on the reaction of an alcohol or thiol with an isocyanate or isothiocyanate, forming the urethane bond as shown in Scheme 1.

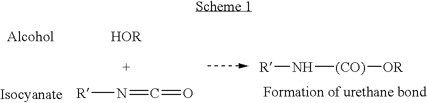

The reaction of a diol and a diisocyanate forms a linear polyurethane, as set forth

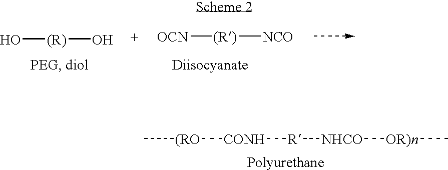

An exemplary T-gel of the invention is prepared by reacting a triol (e.g., TMP), a diol (e.g., PEG) and a diisocyanate (e.g., TDI), as shown in Scheme 3. 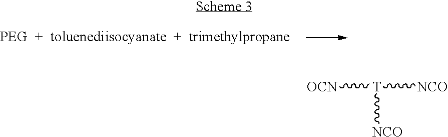
The reaction pathway set forth in Scheme 3 provides isocyanate-terminated polyurethane. Polyurethanes terminated with a variety of reactive functional groups are readily prepared by varying the reactions constituents and/or stoichiometry of the reaction. For example, by adjusting the reaction stoichiometry, a hydroxy-terminated polyurethane is readily prepared.

Cross-linking Monomer

The cross-linking monomer includes at least three moieties, e.g., alcohols, thiols or combinations of these, that can react with an isocyanate or an isothiocyanate to form a urethane bond. The function of the cross-linking monomer is to provide the nucleus of a branching structure on which in the polyurethane can be formed. A preferred cross-linking monomer is a primary or secondary polyol, polythiol or combinations thereof. Preferably the monomer has three or four groups selected from hydroxyls and thiols. An exemplary monomer has an alkyl backbone of four to sixteen carbons or has an aryl nucleus, and generally not more than 20 carbons. Exemplary cross-linking monomers include propane triols, butanetriols, pentanetriols and hexyltriols. Specific examples include trimethylol propane. For a tighter gel, tetraol can be used.

First Monomer

The first monomer includes two reactive moieties selected from the group consisting of a hydroxyl moiety, a thiol moiety or a combinations thereof. The first monomer provides “arms” to the polyurethane polymer. Preferably, the first monomer comprises hydrophilic groups compatible with the formation of a hydrogel upon cross-linking the polyurethane polymers with each other.

In an exemplary embodiment, the first monomer has the formula:
X¹—(CWY¹CH(R)Y²)ₙ—X²
in which the symbols X¹ and X² independently represent OH or SH. The symbols Y¹ and Y² represent moieties that are independently selected from H, halogen, substituted or unsubstituted alkyl, substituted or unsubstituted heteroalkyl, substituted or unsubstituted aryl, substituted or unsubstituted heteroaryl, substituted or unsubstituted heteroarylalkyl, positively charged moieties, negatively charged moieties, metal complexing moieties, metal complexes, hydrophilic moieties, hydrophobic moieties, reactive organic functional groups and combinations thereof. W is H or halogen, e.g., F. R is a member selected from O, S and substituted or unsubstituted alkyl, and the symbol n represents an integer from 1 to 1000.

The first monomer can be a diol, for example, an alkylene glycol, a poly(alkylene glycol), or an aryl, heteroaryl or heterocycloalkyl diol.

In an exemplary embodiment, the first monomer is selected so that the resulting polymer is a hydrophilic polymer. Exemplary first monomers according to this embodiment are non-proteinaceous oligomers or polymers. Suitable hydrophilic polymers include polymers formed from ethylene oxide and propylene oxide polymers (including homopolymers and copolymers), e.g., poly(ethylene glycol), poly(ethylene oxide-co-propylene oxide), and carboxylated poly(ethylene) (e.g., CARBOPOL™). Other exemplary first monomers include poly(phosphazene) species, and polysaccharides, poly(amino acids), and blends of hydrophilic polymers.

In a preferred embodiment, the first monomer is a poly(alkylene oxide), such as polyethylene glycol or polypropylene glycol having molecular weights from about 200 to about 20,000, preferably about 200 to about 4000.

Second Monomer

The second monomer includes at least two reactive moieties selected from the group consisting of an isocyanate moiety, an isothiocyanate moiety or a combination thereof. The second monomer couples the first monomer to the cross-linking monomer through urethane bonds, and provides reactive isocyanate groups at the ends of polyurethane branches that can engage in a cross-linking reaction with other polyurethane units during the curing process so as to produce the hydrogel.

An exemplary second monomer has the formula:
Z¹═C═N—R¹—N═C=Z²
wherein the symbol R¹ represents a moiety that is selected from substituted or unsubstituted alkyl, substituted or unsubstituted heteroalkyl, substituted or unsubstituted aryl, substituted or unsubstituted heteroaryl, and substituted or unsubstituted heterocycloalkyl moieties. Z¹ and Z² are independently selected from O and S. In the formula above, when R¹ is alkyl or aryl, it is preferably selected from substituted or unsubstituted C₄-C₂₂ alkyl (e.g., phoshotidyl glycerol) and substituted or unsubstituted C₆-C₁₂ aryl. More preferably, R¹ is a member selected from substituted or unsubstituted phenyl, substituted or unsubstituted cyclohexyl, and substituted or unsubstituted alkyl.

Examples of suitable first monomers include toluenediisocyanate, cyclohexyldiisocyanate, butyldiisocyanate and hexyldiisocyanate.

Functional Monomer

Exemplary hydrogels of this invention are functionalized with one or more group conveniently designated as a binding functionality or an EAM or SEND functionality. Generally, these functionalities are incorporated into the T-gel through functional monomers that include the desired functionality and a moiety that reacts with an isocyanate group to form a covalent bond, e.g., a primary or secondary alcohol, thiol or amine. Generally, the functional monomer will be small enough so as to not interfere with T-gel or hydrogel formation. For example, the functional monomer can have a molecular weight between about 50 Daltons and 2000 Daltons. In certain instances, a large moiety, such as heparin, can be used.

Binding Functionalities

Binding functionalities fall into two classes: Reactive functionalities that form a covalent bond with the target, and adsorbent functionalities, that form a non-covalent bond with the target.

Reactive Functionalities

Reactive functional groups are useful for attaching other molecules to the hydrogel. For example, one may want to attach biomolecules, such as polypeptides, nucleic acids, carbohydrates or lipids to the hydrogel. Exemplary reactive functional groups include:
(a) carboxyl derivatives such as N-hydroxysuccinimide esters, N-hydroxybenztriazole esters, acid halides, acyl imidazoles, thioesters, p-nitrophenyl esters, alkyl, alkenyl, alkynyl and aromatic esters;
(b) haloalkyl groups wherein the halide can be later displaced with a nucleophilic group such as, for example, a bromoacetyl group;
(c) aldehyde or ketone groups such that subsequent derivatization is possible via formation of carbonyl derivatives such as, for example, imines, hydrazones, semicarbazones or oximes, or via such mechanisms as Grignard addition or alkyllithium addition;
(d) sulfonyl halide groups for subsequent reaction with amines, for example, to form sulfonamides;
(e) reactive thiol groups, which can react with disulfides on proteins, including 2-mercaptopyridines and orthopydinyl disulfides;
(f) sulfhydryl groups, which can be, for example, acylated or alkylated;
(g) alkenes, which can undergo, for example, Michael addition, etc (e.g., maleimide);
(h) epoxides, which can react with nucleophiles, for example, amines and hydroxyl compounds;
(i) hydrazine groups, which react with sugars and glycoproteins;
(j) vinyl sulfones;
(k) activated carbonyl groups such as.

The reactive functional groups can be chosen such that they do not participate in, or interfere with reactions in which they are not intended to participate in. Alternatively, the reactive functional group can be protected from participating in the reaction by the presence of a protecting group. Those of skill in the art will understand how to protect a particular functional group from interfering with a chosen set of reaction conditions. For examples of useful protecting groups, See, Greene et al., PROTECTIVE GROUPS IN ORGANIC SYNTHESIS, John Wiley & Sons, New York, 1991.

Those of skill in the art understand that the reactive functional groups discussed herein represent only a subset of functional groups that are useful in assembling the chips of the invention. Moreover, those of skill understand that the reactive functional groups are also of use as components of the functionalized film and the linker arms.

As shown in Table 1, an isocyanate polymer of the invention allows access to polymers having an array of reactive functionalities for immobilization of binding functionalities, EAM, linker arms, binding functionality- or EAM-linker arm cassettes and analytes.

TABLE 1
PROTEIN BIOCHIP SELECTED REACTIVE CHEMISTRY

| Functional Group | Co-reactant | nucleophiles | pH |
|---|---|---|---|
| Imidazocarbonyl | NA | amine | 7-8 |
| Epoxy | NA | amine | 8-9 |
| Aldehyde | NaCNBH3 | amine | 6-9 |
| Thiol | NA | disulfide | 5.5-9 |
| Thiol | PDS | thiol | 6-8 |
| NHS | NA | amine | 6-8 |
| NHSA | NA | amine | 6-7 |
| NHM | NA | thiol | 6.5 |
| | | | 9 |
| Iodoacetyl | NA | amine | 9 |
| | | thiol | 9 |
| | | Sulfide | 9 |
| Iodoacetyl | Methionine | amine | 8.5-8.8 |
| Vinylsulfone | NA | Thiol | 7 |
| PNP | NA | amine | 8-9 |

Hepes: 4-(2-hydroxyethyl)-piperazine-1-ethanesulfonic acid

Exemplary reactive functional monomers are imidazole, phenylcarboxyethanol, N-hydroxysuccinimide, N-hydroxymaleimide, cystamine/DTT, glycidol, p-nitrophenyl methylol carbonate, benzotriazoyl methylol carbonate, MeSCH₂ CH₂OH, Ellman's reagent (4-nitro-3-carboxylic acid)disulfide and O-pyridinyl-disulfide.

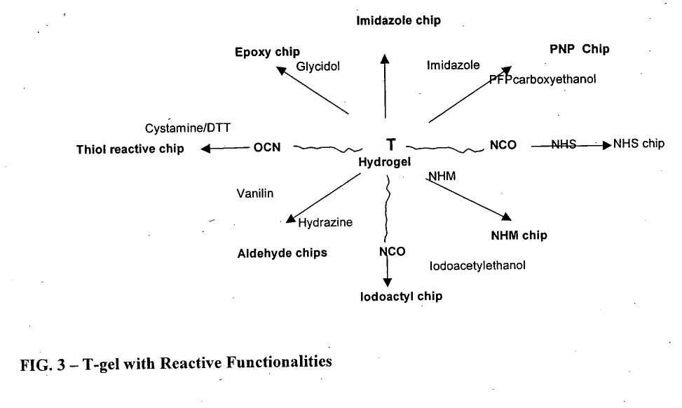

Selected pathways available for functionalizing the activated polyurethane of the invention with a reactive group are shown in FIG. 3.

Adsorbent Functionalities

Binding functionalities (which also can be attached through reactive functionalities) are useful for capturing analytes from a sample for further analysis. Binding functionalities may be grouped into two classes—biospecific binding groups and chromatographic binding groups.

Binding functionalities can be chromatographic or biospecific. Chromatographic binding functionalities bind substances via charge-charge, hydrophilic-hydrophilic, hydrophobic-hydrophobic, van der Waals interactions and combinations thereof.

Biospecific binding functionalities generally involve complementary 3-dimensional structures involving one or more of the above interactions. Examples of combinations of biospecific interactions include, but are not limited to, antigens with corresponding antibody molecules, a nucleic acid sequence with its complementary sequence, effector molecules with receptor molecules, enzymes with inhibitors, sugar chain-containing compounds with lectins, an antibody molecule with another antibody molecule specific for the former antibody, receptor molecules with corresponding antibody molecules and the like combinations. Other examples of the specific binding substances include a chemically biotin-modified antibody molecule or polynucleotide with avidin, an avidin-bound antibody molecule with biotin and the like combinations. Biospecific functionalities are generally produced by attaching the biospecific moiety through a reactive moiety, as above.

In an exemplary embodiment, the binding functionality monomer includes a binding functionality that is selected the group consisting of a positively charged moiety, a negatively charged moiety, an anion exchange moiety, a cation exchange moiety, a metal ion complexing moiety, a metal complex, a polar moiety, a hydrophobic moiety. Further exemplary binding functionalities include, an amino acid, a dye, a carbohydrate, a nucleic acid, a polypeptide, a lipid (e.g., a phosphotidyl choline), and a sugar.

Ion exchange moieties of use as binding functionalities in the polymers of the invention are, e.g., diethylaminoethyl, triethylamine, sulfonate, tetraalkylammonium salts and carboxylate.

In an exemplary embodiment, the binding functionality is a polyaminocarboxylate chelating agent such as ethylenediaminetetraacetic acid (EDTA) or diethylenetriaminepentaacetic acid (DTPA), which is attached to an amine on the substrate, or spacer arm, by utilizing the commercially available dianhydride (Aldrich Chemical Co., Milwaukee, Wis.). When complexed with a metal ion, the metal chelate binds to tagged species, such as polyhistidyl-tagged proteins, which can be used to recognize and bind target species. Alternatively, the metal ion itself, or a species complexing the metal ion can be the target.

Metal ion complexing moieties include, but are not limited to N-hydroxyethylethylenediaminoe-triacetic acid (NTA), N,N-bis(carboxymethyl)-L-lysine, iminodiacetic acid, aminohydroxamic acid, salicylaldehyde, 8-hydroxy-quinoline, N,N,N′-tris(carboxytrimethyl)ethanolamine, and EDTA, DTPA and N-(2-pyridylmethyl) aminoacetate. The metal ion complexing agents can complex any useful metal ion, e.g., copper, iron, nickel, cobalt, gallium and zinc.

The organic functional group can be a component of a small organic molecule with the ability to specifically recognize an analyte molecule. Exemplary small organic molecules include, but are not limited to, amino acids, heparin, biotins, avidin, streptavidin carbohydrates, glutathiones, nucleotides and nucleic acids.

In another exemplary embodiment, the binding functionality is a biomolecule, e.g., a natural or synthetic peptide, antibody, nucleic acid, saccharide, lectin, member of a receptor/ligand binding pair, antigen, cell or a combination thereof. Thus, in an exemplary embodiment, the binding functionality is an antibody raised against a target or against a species that is structurally analogous to a target. In another exemplary embodiment, the binding functionality is avidin, or a derivative thereof, which binds to a biotinylated analogue of the target. In still another exemplary embodiment, the binding functionality is a nucleic acid, which binds to single- or double-stranded nucleic acid target having a sequence complementary to that of the binding functionality.

In another exemplary embodiment, the chip of this invention is an oligonucleotide array in which the binding functionality at each addressable location in the array comprises a nucleic acid having a particular nucleotide sequence. In particular, the array can comprise oligonucleotides. For example, the oligonucleotides can be selected so as to cover the sequence of a particular gene of interest. Alternatively, the array can comprise cDNA or EST sequences useful for expression profiling.

In a further preferred embodiment, the binding functionality is selected from nucleic acid species, such as aptamers and aptazymes that recognize specific targets.

In another exemplary embodiment, the binding functionality is a drug moiety or a pharmacophore derived from a drug moiety. The drug moieties can be agents already accepted for clinical use or they can be drugs whose use is experimental, or whose activity or mechanism of action is under investigation. The drug moieties can have a proven action in a given disease state or can be only hypothesized to show desirable action in a given disease state. In a preferred embodiment, the drug moieties are compounds, which are being screened for their ability to interact with a target of choice. As such, drug moieties, which are useful in practicing the instant invention include drugs from a broad range of drug classes having a variety of pharmacological activities.

Exemplary hydrophobic adsorbent functional monomers include CH₃(CH₂)₁₇OH, 1-octadecanol, 1-docosanol, perfluorinated polyethyleneglycol (Sovay, USA).

Exemplary hydrophilic adsorbent functional monomers include polyvinyl alcohol) and polyvinylpyrolidone.

Exemplary anion exchange adsorbent functional monomers include 3-chloro-2-hydroxypropyl trimethylammonium chloride and 2-hydroethyl-N-methylpyridinium chloride.

Exemplary cation exchange adsorbent functional monomers include 1,4-butanediol-2-sulfonic acid, 3,5-dimethyl-o-benzenesulfonic acid, dihydroxybenzoic acid and dimethylol acetic acid.

Exemplary metal chelate adsorbent functional monomers include N-hydroxyethylethylenediamino-triacetic acid (NTA), N,N-bis(carboxymethyl)-L-lysine, aminohydroxamic acid, salicylaldehyde, 8-hydroxy-quinoline, N,N,N′-tris(carboxytrimethyl)ethanolamine, and N-(2-pyridylmethyl)aminoacetate. The addition of a solution of metal ions, such as copper, nickel, zinc, iron and gallium functionalizes the gel.

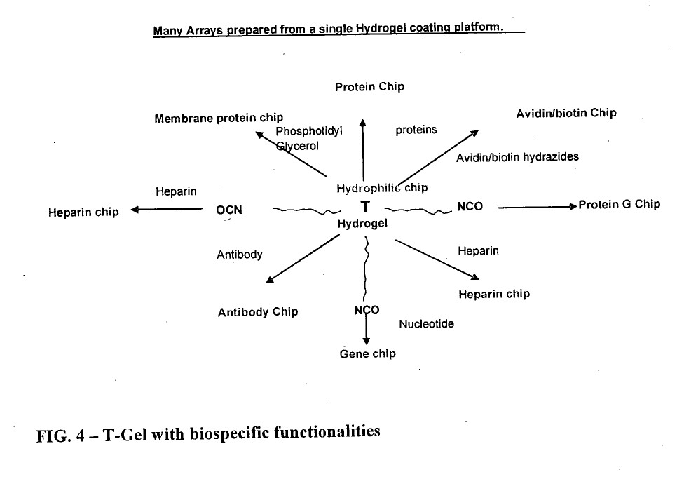

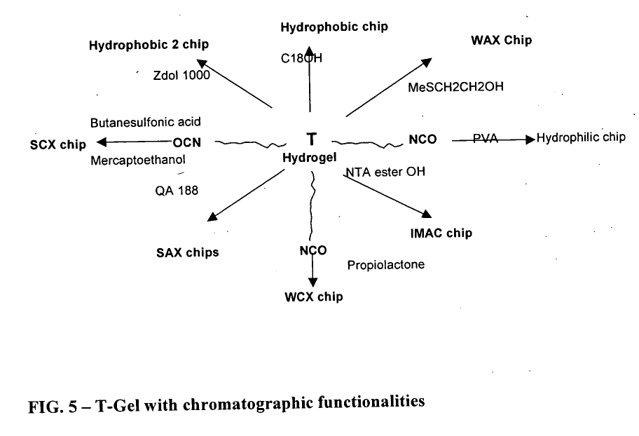

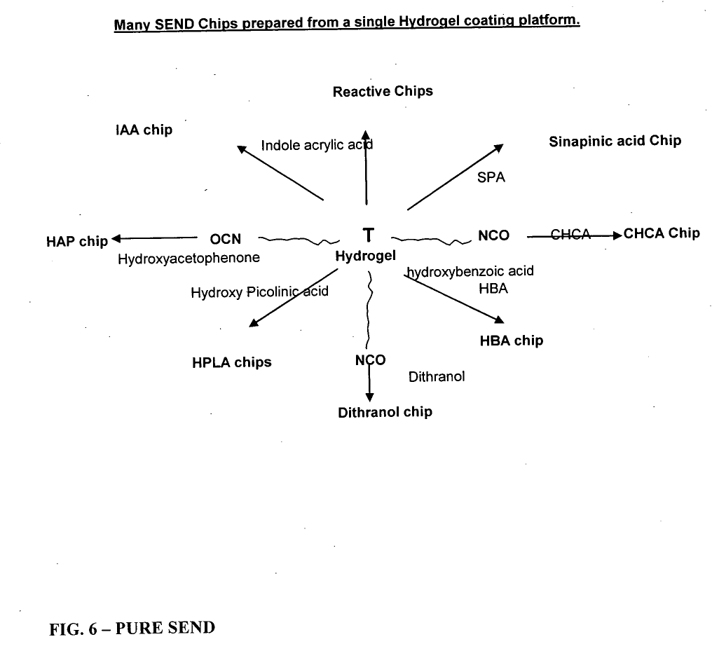

Exemplary reaction pathways for preparing polyurethanes with adsorbent functionalities are set forth in FIG. 4 and FIG. 5.

EAM Functionalities

EAM (energy absorbing molecule) functionalities are useful for promoting desorption and ionization of analyte into the gas phase during laser desorption/ionization processes. The EAM monomer comprises a photo-reactive moiety as a functional group. The photo-reactive moiety preferably includes a nucleus or prosthetic group that specifically absorbs photo-radiation from a laser source. The photo-reactive groups absorbs energy from a high fluence source to generate thermal energy, and transfers the thermal energy to promote desorption and ionization of an analyte in operative contact with the polyurethane. In the case of UV laser desorption, the EAM monomer preferably includes an aryl nucleus that electronically absorbs UV photo-irradiation. In the case of IR laser desorption, the EAM monomer preferably includes an aryl nucleus or a group that preferably absorbs the IR radiation through direct vibrational resonance or in slight off-resonance fashion. A UV photo-reactive moiety can be selected from benzoic acid (e.g., 2,5 di-hydroxybenzoic acid), cinnamic acid (e.g., α-cyano-4-hydroxycinnamic acid), acetophenone, quinone, vanillic acid, caffeic acid, nicotinic acid, sinapinic acid pyridine, ferrulic acid, 3-amino-quinoline and derivatives thereof. An IR photo-reacitve moiety can be selected from benzoic acid (e.g., 2,5 di-hydroxybenzoic acid), cinnamic acid (e.g., α-cyano-4-hydroxycinnamic acid), acetophenone (e.g. 2,4,6-trihyroxyacetophenone and 2,6-dihyroxyacetophenone) caffeic acid, ferrulic acid, sinapinic acid 3-amino-quinoline and derivatives thereof.

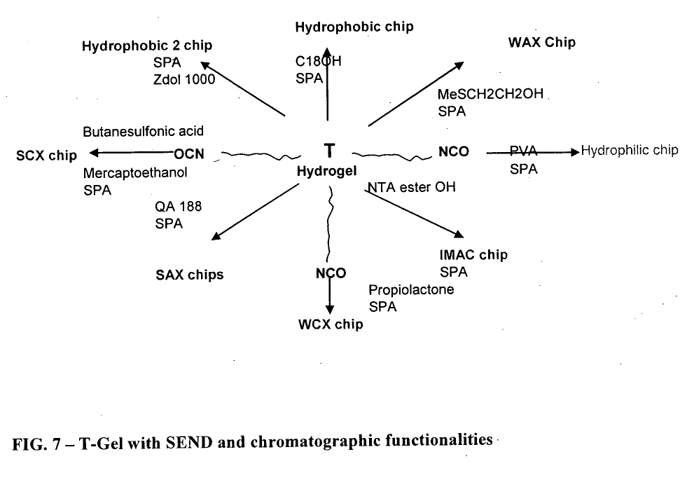

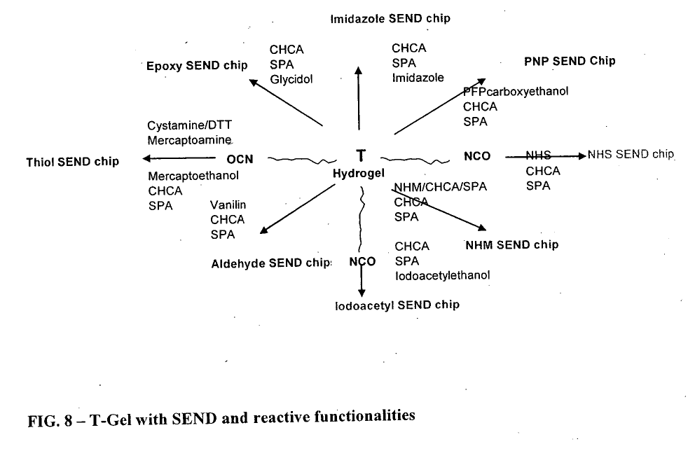

FIG. 7 and FIG. 8 set forth exemplary reaction pathways for producing polyurethane bearing an EAM and a binding functionality.

Preparation of Polyurethane Polymer

The monomers above are assembled into a polyurethane polymer of this invention. The monomers are combined in selected proportions and subjected to polymerization reaction conditions so that the bulk of the polymers produced comprise one cross-linking monomer with an “arm” attached to each reactive group (e.g., an alcohol or a thiol). An exemplary structure, when the cross-linking monomer is a triol, is: FnM-SM-FM-SM-CRM-SM-FM-SM-NCO)₂, where CRM is the cross-linking monomer, SM is the second monomer, FM is the first monomer and FnM is the functional monomer. Thus, the cross-linking monomer, the second monomer and the first monomer are attached to one another through urethane bonds. Furthermore, conditions are optimally set so that the polyurethane polymer comprises at least two isocyanate groups (NCO) at the ends of the arms which can engage in a polymerization reaction upon curing to produce the hydrogel. Again, when the cross-linking moiety is a triol, the polyurethane polymer will be a “T-gel,” and if the cross-linking moiety is a tetra-ol, the polyurethane polymer will be a “+-gel.” Exemplary ratios of triol:di-isocyanate:diol (i.e., CRM:SM:FM) include from about 1:5-20:5-50 to about about 1:7:3. The ratio of the triol to the functional monomer is preferably between 1:0.1-3.

The functional monomer can be incorporated into they hydrogel at any stage of its production. For example, one can polymerize the first monomer, second monomer, cross-linking monomer and functional monomer together to create a functionalized gel in one step. It may, however, be more convenient to create a functionalized gel by reacting the functional monomer with already formed gel. In this way, one can employ a single batch of polyurethane gel to make many differently functionalized gels. This methods has the advantage of improved consistency of chip surface composition.

In another embodiment, one can functionalize the gel by adding the functional monomer before, during or after the curing process. The choice can depend on the nature of the hydrogel and the functional monomer. Preferably, if the functionality will survive the polymerization reaction, the functional monomer is incorporated into the T-gel during T-gel formation. Highly reactive groups, such as hydrazine, will tend to cause cross-linking of the T-gel. Therefore, they it is preferred to add functional monomers with such groups to the T-gel mixture upon curing. The amount of unreacted isocyanate function can be controlled by cure time. The hydrazine can be then incorporated into the gel by reacting with the unreacted isocyanate.

In another exemplary embodiment, the reactive polyurethane polymers are prepared by reacting a terminal isocyanate of a T-Gel with a molecule with a protein capturing functional group and an alcohol, thiol, or amine group. When the reactive group is an amine or an alcohol, it reacts with the bulk (e.g., approximately 50%) of the terminal isocyanate groups, forming urea and urethane bonds, respectively. The remaining isocyanate groups (about 50%) are available to form cross-links with a group on the surface of a substrate onto which the polymer is layered. For example, the isocyanate groups react readily with silanol moieties on a glass surface, immobilizing the polymer thereon. In another exemplary embodiment, the isocyanates react with NH groups on an organic polymer backbone, thereby binding the polyurethane to the amine-containing organic polymer.

The Devices

The devices of this invention comprise a solid support having a surface and a polyurethane-based hydrogel attached to the surface. A preferred way of making the devices of this invention involves polymerizing the polyurethane polymer units described above through curing on the surface of the solid support. More particularly, curing causes a reaction between the free isocyanates at the ends of the arms of the polyurethane polymer unit to react with the urethane bonds in the arms of the polyurethane polymer unit. The reaction results in the formation of a covalent urea bond that couples one polyurethane polymer unit to another. Because the polyurethane polymer units are constructed to possess a plurality of free isocyanate moieties, the coupling reaction results in a cross-linked hydrogel. As discussed above, the hydrogel may already be functionalized, or may be functionalized after cross-linking through remaining free isocyantes. Furthermore, the attachment of the hydrogel to the solid support can be covalent by the provision on the surface of reactive groups, such as hydroxyls, thiols or amines that can form a covalent bond with the free isocyanate groups.

The devices of this invention may be in the form of chips, chromatographic materials or membranes, depending upon the nature of the solid substrate and the intended use. The following section is generally applicable to each device of the invention. In selected devices of the invention (e.g., chips, chromatographic supports, membranes), the functionalized film is immobilized on a substrate, either directly or through linker arm arms that are interposed between the substrate and the functionalized film. The nature and intended use of the device influences the configuration of the substrate. For example, a chip of the invention is typically based upon a planar substrate format. In contrast, a chromatographic support of the invention generally makes us of a spherical or approximately spherical substrate, while a membrane of the invention is formed using a porous substrate.

In general, the hydrogel is prepared by contacting the T-gel or functionalized T-gel with the surface and heating the material to cause polymerization. This method is referred to as “curing.” Curing can be accomplished by heating the material for between about 30 minutes and about 5 hours at a temperature between about 20° C. and about 200° C. (preferably between about 50° and about 100° C. in an inert gas environment). In a presently preferred embodiment, the gel is derivatized with the functional monomer prior to curing.

When the solid support is a chip, the T-gel can be applied to the surface by an useful method, e.g., spotting (to discrete locations), spin coating (to cover the entire surface) or dipping. The thickness of the gel depends on the intended use of the gel. For surface scanning techniques, such as surface plasmon resonance or diffraction grating coupled optical waveguide biosensors, the gel is preferably between about 50 nm and about 200 nm. For methods such as SELDI mass spectrometry, the thickness is preferably from about 50 nm to about 10 microns.

Solid Support Materials

Exemplary substrate materials include, but are not limited to, inorganic crystals, inorganic glasses, inorganic oxides, metals, organic polymers and combinations thereof. Inorganic glasses and crystals of use in the substrate include, but are not limited to, LiF, NaF, NaCl, KBr, KI, CaF₂, MgF₂, HgF₂, BN, AsS₃, ZnS, Si₃N₄, AIN and the like. The crystals and glasses can be prepared by art standard techniques. See, for example, Goodman, CRYSTAL GROWTH THEORY AND TECHNIQUES, Plenum Press, New York 1974. Alternatively, the crystals can be purchased commercially (e.g., Fischer Scientific). Inorganic oxides of use in the present invention include, but are not limited to, Cs₂O, Mg(OH)₂, TiO₂, ZrO₂, CeO₂, Y₂O₃, Cr₂O₃, Fe₂O₃, NiO, ZnO, Ta₂O₅, Al₂O₃, SiO₂ (glass), quartz, In₂O₃, SnO₂, PbO₂ and the like. Metals of use in the substrates of the invention include, but are not limited to, gold, silver, platinum, palladium, nickel, copper and alloys and composites of these metals.

Organic polymers that form useful substrates include, for example, polyalkenes (e.g., polyethylene, polyisobutene, polybutadiene), polyacrylics (e.g., polyacrylate, polymethyl methacrylate, polycyanoacrylate), polyvinyls (e.g., polyvinyl alcohol, polyvinyl acetate, polyvinyl butyral, polyvinyl chloride), polystyrenes, polycarbonates, polyesters, polyurethanes, polyamides, polyimides, polysulfone, polysiloxanes, polyheterocycles, cellulose derivative (e.g., methyl cellulose, cellulose acetate, nitrocellulose), polysilanes, fluorinated polymers, epoxies, polyethers and phenolic resins.

In a preferred embodiment, the substrate material is substantially non-reactive with the target, thus preventing non-specific binding between the substrate and the target or other components of an assay mixture. Methods of coating substrates with materials to prevent non-specific binding are generally known in the art. Exemplary coating agents include, but are not limited to cellulose, bovine serum albumin, and poly(ethyleneglycol). The proper coating agent for a particular application will be apparent to one of skill in the art.

Linker Arms

The hydrogel of the invention is attached to the surface of the solid support by a variety of means. The interaction between the hydrogel and the surface, which anchors the polymer to the surface can be a covalent, electrostatic, ionic, hydrogen bonding, hydrophobic-hydrophobic, or hydrophilic-hydrophilic interaction. When the interaction is non-covalent, it is referred to herein as “physical adhesion.”

The following section is generally applicable to each device of the invention. In certain embodiments, the device incorporates a linker arm between the substrate and the polyurethane. The layer of linker arms is of any composition and configuration useful to immobilize the functionalized film. The linker arms are bound to and immobilized on the substrate. The linker arms also have one or more groups that interact with the functionalized film.

The polyurethane film is attached to the linker arm layer by one of many interaction modalities with which one of skill in the art is familiar. Representative modalities include, but are not limited to, covalent attachment, attachment via polymer entanglement and electrostatic attachment.

In a preferred embodiment, the hydrogel can be covalently bound to the chip by providing the chip with surface moieties that chemically couple with a reactive group on of the hydrogel, e.g., free isocyanates, alcohols, thiols or amines. Thus, for example, the substrate can have a glass (silicon dioxide) coating that provides hydroxyl groups for reaction with an isocyanate. Alternatively, the surface can have attached amino alkyl silane groups which provide amine groups.

In another embodiment, the hydrogel is attached to the surface through a linker arm, which is attached to both the surface and the hydrogel. The linker arms can be selected from synthetic and biological polymers, as well as small molecule linkers (e.g., alkyl, heteroalkyl, etc.). A fully assembled linker can be coupled to the substrate. Alternatively, the linker arms can be assembled on the substrate by coupling together linker arm components using a functional group on the substrate as the origin of linker arm synthesis. The point of attachment to either the substrate or polyurethane is preferably at a terminus of the linker arm, but can also be an internal site. The linker arm can be a linear molecular moiety or it can be branched. The linker arms on a substrate may be independent or they may be crosslinked with one another. In one embodiment, the collection of linker arms forms a “brush polymer,” that is, a collection of molecular strands, each independently attached to the substrate.

Exemplary synthetic linker species useful in the chips of the present invention include both organic and inorganic polymers and may be formed from any compound, which will support the immobilization of the functionalized film. For example, synthetic polymer ion-exchange resins such as poly(phenol-formaldehyde), polyacrylic-, or polymethacrylic-acid or nitrile, amine-epichlorohydrin copolymers, graft polymers of styrene on polyethylene or polypropylene, poly(2-chloromethyl-1,3-butadiene), poly(vinylaromatic) resins such as those derived from styrene, α-methylstyrene, chlorostyrene, chloromethylstyrene, vinyltoluene, vinylnaphthalene or vinylpyridine, corresponding esters of methacrylic acid, styrene, vinyltoluene, vinylnaphthalene, and similar unsaturated monomers, monovinylidene monomers including the monovinylidine ring-containing nitrogen heterocyclic compounds and copolymers of the above monomers are suitable.

In another embodiment, the linker is a lipophilic polymer. Exemplary lipophilic polymers are polyester (e.g., poly(lactide), poly(caprolactone), poly(glycolide), poly(6-valerolactone), and copolymers containing two or more distinct repeating units found in these named polyesters), poly(ethylene-co-vinylacetate), poly(siloxane), poly(butyrolactone), and poly(urethane).

Chips

This invention contemplates devices in which the surface of a substrate is coated with the monomeric or polymeric complexes of this invention. The complexes can be bound to the surface by any means, including covalent or non-covalent chemical bonding, or simply physical attachment by applying the complex to the substrate surface where it sticks. Depending on the nature of the substrate, the devices of this invention can come in the form of chips, resins (e.g., beads), microtiter plates or membranes.

a. Substrate

In selected devices of the invention (e.g., chips, chromatographic supports, microtiter plates, membranes), the complex is immobilized on a substrate, either directly or through linker arms that are interposed between the substrate and the adsorbent film. The nature and intended use of the device influences the configuration of the substrate. For example, a chip of the invention is typically based upon a planar substrate format. In contrast, a chromatographic support of the invention generally makes use of a spherical or approximately spherical substrate, while a membrane of the invention is formed using a porous substrate. A microtiter plate is generally a plastic article of manufacture comprising wells in which reactions can be performed.

b. Chip

Exemplary chips of the invention are formed using a planar substrate. The complex is applied directly to the substrate or is bound to an anchor moiety that is bound to the substrate surface, or to a feature on the substrate surface, such as a region that is raised (e.g., island) or depressed (e.g., a well, trough, etc.).

The gel of the invention is generally attached to the chip substrate. The interaction between the polymer and the substrate can be a covalent, electrostatic, ionic, hydrogen bonding, hydrophobic-hydrophobic, hydrophilic-hydrophilic interaction or physisorption or physical adhesion.

Substrates that are useful in practicing the present invention can be made of any stable material, or combination of materials. Moreover, useful substrates can be configured to have any convenient geometry or combination of structural features. The substrates can be either rigid or flexible and can be either optically transparent or optically opaque. The substrates can also be electrical insulators, conductors or semiconductors. When the sample to be applied to the chip is water based, the substrate preferable is water insoluble.

In a preferred embodiment, the substrate material is essentially non-reactive with the analyte, thus preventing non-specific binding between the substrate and the analyte or other components of an assay mixture. Methods of coating substrates with materials to prevent non-specific binding are generally known in the art. Exemplary coating agents include, but are not limited to cellulose, bovine serum albumin, and poly(ethylene glycol). The proper coating agent for a particular application will be apparent to one of skill in the art.

In an exemplary embodiment, the substrate includes an aluminum support that is coated with a layer of silicon dioxide. In yet a further preferred embodiment, the silicon dioxide layer is from about 1000-3000 Å in thickness. In other embodiments, the substrate comprises a polymeric material, such as cellulose or a plastic.

In preferred embodiments, the chip functions as a probe for a mass spectrometer.

In a preferred embodiment, the functionalized film of a chip of the invention is configured such that detection of the immobilized analyte does not require elution, recovery, amplification, or labeling of the target analyte. In another embodiment, the detection of one or more molecular recognition events, at one or more locations within the addressable functionalized film, does not require removal or consumption of more than a small fraction of the total adsorbent-analyte complex. Thus, the unused portion can be interrogated further after one or more “secondary processing” events conducted directly in situ (i.e., within the boundary of the addressable location) for the purpose of structure and function elucidation, including further assembly or disassembly, modification, or amplification (directly or indirectly).

The surface of a substrate of use in practicing the present invention can be smooth, rough and/or patterned. The surface can be engineered by the use of mechanical and/or chemical techniques. For example, the surface can be roughened or patterned by rubbing, etching, grooving, stretching, and the oblique deposition of metal films. The substrate can be patterned using techniques such as photolithography (Kleinfield et al., J. Neurosci. 8: 4098-120 (1998)), photoetching, chemical etching and microcontact printing (Kumar et al., Langmuir 10: 1498-511 (1994)). Other techniques for forming patterns on a substrate will be readily apparent to those of skill in the art.

The size and complexity of the pattern on the substrate is controlled by the resolution of the technique utilized and the purpose for which the pattern is intended. For example, using microcontact printing, features as small as 200 nm have been layered onto a substrate. See, Xia, Y.; Whitesides, G., J. Am. Chem. Soc. 117: 3274-75 (1995). Similarly, using photolithography, patterns with features as small as 1 μm have been produced. See, Hickman et al., J. Vac. Sci. Technol. 12: 607-16 (1994). Patterns that are useful in the present invention include those which comprise features such as wells, enclosures, partitions, recesses, inlets, outlets, channels, troughs, diffraction gratings and the like.

In an exemplary embodiment, the patterning is used to produce a substrate having a plurality of adjacent addressable features, wherein each of the features is separately identifiable by a detection means. In another exemplary embodiment, an addressable feature does not fluidically communicate with other adjacent features. Thus, an analyte, or other substance, placed in a particular feature remains essentially confined to that feature. In another preferred embodiment, the patterning allows the creation of channels through the device whereby fluids can enter and/or exit the device.

Using recognized techniques, substrates with patterns having regions of different chemical characteristics can be produced. Thus, for example, an array of adjacent, isolated features is created by varying the hydrophobicity/hydrophilicity, charge or other chemical characteristic of a pattern constituent. For example, hydrophilic compounds can be confined to individual hydrophilic features by patterning “walls” between the adjacent features using hydrophobic materials. Similarly, positively or negatively charged compounds can be confined to features having “walls” made of compounds with charges similar to those of the confined compounds. Similar substrate configurations are also accessible through microprinting a layer with the desired characteristics directly onto the substrate. See, Mrkish, M.; Whitesides, G. M., Ann. Rev. Biophys. Biomol. Struct. 25:55-78 (1996).

The specificity and multiplexing capacity of the chips of the invention is improved by incorporating spatial encoding (e.g., spotted microarrays) into the chip substrate. Spatial encoding can be introduced into each of the chips of the invention. In an exemplary embodiment, binding functionalities for different analytes can be arrayed across the chip surface, allowing specific data codes (e.g., target-binding functionality specificity) to be reused in each location. In this case, the array location is an additional encoding parameter, allowing the detection of a virtually unlimited number of different analytes.

In the embodiments of the invention in which spatial encoding is utilized, they preferably utilize a spatially encoded array comprising m binding functionalities distributed over m regions of the substrate. Each of the m binding functionalities can be a different functionality or the same functionality, or different functionalities can be arranged in patterns on the surface. For example, in the case of matrix array of addressable locations, all the locations in a single row or column can have the same binding functionality. The m binding functionalities are preferably patterned on the substrate in a manner that allows the identity of each of the m locations to be ascertained. In another embodiment, the m binding functionalities are ordered in a p by q matrix of (p×q) discrete locations, wherein each of the (p×q) locations has bound thereto at least one of the m binding functionalities. The microarray can be patterned from essentially any type of binding functionality.

Mass Spectrometry Probe

In preferred embodiments the chip of this invention is designed in the form of a probe for a gas phase ion spectrometer, such as a mass spectrometry probe. To facilitate its being positioned in a sample chamber of a mass spectrometer, the substrate of the chip is generally configured to comprise means that engage a complementary structure within the interface. The term “positioned” is generally understood to mean that the chip can be moved into a position within the sample chamber in which it resides in appropriate alignment with the energy source for the duration of a particular desorption/ionization cycle. There are many commercially available laser desorption/ionization mass spectrometers. Vendors include Ciphergen Biosystems, Inc., Waters, Micromass, MDS, Shimadzu, Applied Biosystems and Bruker Biosciences.

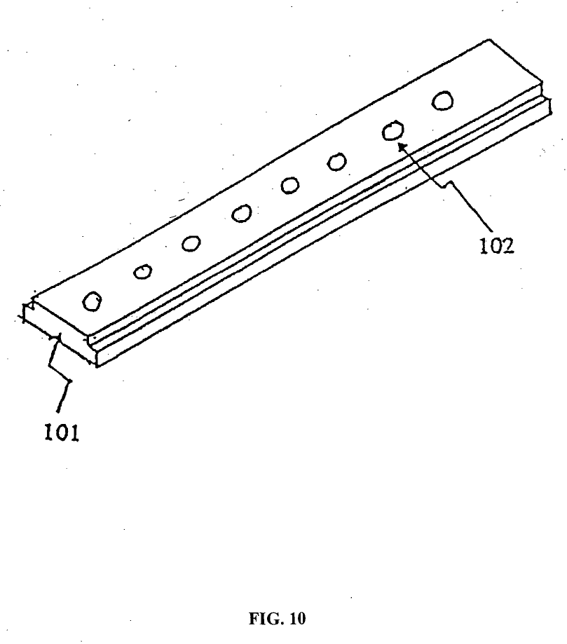

An exemplary structure according to this description is a chip that includes means for slidably engaging a groove in an interface, such as that used in the Ciphergen probes (FIG. 10). In this figure, the means to position the probe in the sample chamber is integral to substrate 101, which includes a lip 102 that engages a complementary receiving structure in the probe.

In another example, the probe is round and is typically attached to a holder/actuator using a magnetic coupler. The target is then pushed into a repeller and makes intimate contact to insure positional and electrical certainty.

Other probes are rectangular and they either marry directly to a carrier using a magnetic coupling or physically attach to a secondary carrier using pins or latches. The secondary carrier then magnetically couples to a sample actuator. This approach is generally used by systems which have autoloader capability and the actuator is generally a classical x,y 2-d stage.

In yet another exemplary embodiment, the probe is a barrel. The barrel was used to support gel pieces or blots. By rotating and moving in the vertical plane, a 2-d stage is created.

Still a further exemplary embodiment the probe is a disk. The disk is rotated and moved in either a vertical or horizontal position to create an r-theta stage. Such disks are typically engaged using either magnetic or compression couplers.

Chromatographic Supports

In an exemplary embodiment, the polyurethane of the invention is used to form a chromatographic support. A layer of the polyurethane is used to coat a particulate substrate. Particulate substrates that are useful in practicing the present invention can be made of practically any physicochemically stable material. Useful particulate substrates are not limited to a size or range of sizes. The choice of an appropriate particle size for a given application will be apparent to those of skill in the art. In certain preferred embodiments, the substrate has a diameter of from about 1 micrometer to about 1000 micrometers. In other preferred embodiments, the substrate has a diameter of from about 50 micrometers to about 500 micrometers. Many commercially available polymers and resins can also be used in practicing the present invention.

In an exemplary embodiment, the chromatographic support is designed for methods that involve “capture” of an analyte. As used herein, the term “capture” refers to an interaction between a group on the material of the invention and a complementary group on an analyte. The interaction can be either reversible or irreversible. Molecules can be captured from a variety of milieus, including pure liquids, solutions, gases, vapors and the like. This embodiment of the invention can be used for a broad range of applications including, for example, chromatography (e.g., affinity, gas, ion exchange, reverse-phase, normal-phase), assays, proton sponges, catalysis, concentration of trace materials and the like. Further, the capturing can be an end in itself (e.g., removing a contaminant from a mixture) or it can be a step in a multi-step process (e.g., recovering an analyte from a mixture). An example of a method using capture is affinity chromatography.

The particles of the invention can also be used as a solid support for a variety of syntheses. The particles are useful supports for synthesis of small organic molecules, polymers, nucleic acids, peptides and the like. See, for example, Kaldor et al., “Synthetic Organic Chemistry on Solid Support,” In, COMBINATORIAL CHEMISTRY AND MOLECULAR DIVERSITY IN DRUG DISCOVERY, Gordon et al., Eds., Wiley-Liss, New York, 1998.

Membranes

In an exemplary embodiment, the polyurethane of the invention is used to form a membrane. A layer of the polyurethane is used to coat a porous substrate. The invention provides easily prepared and characterized membranes that are capable of presenting a wide range of binding functionalities (ionic groups, metal, complexing agents, biomolecules, and the like), pore sizes, surface charges and surface hydrophilicity/hydrophobicity. Because the porous materials can be shaped, bent or molded into virtually any desired shape, whether planar or curved, the membranes can be prepared in a wide range of forms. The choice of appropriate shape and size will depend on the particular application for the materials of the invention and is well within the abilities of those of skill in the art.

In addition to size and shape, the pore size and pore density of the membranes can be selected from a wide array of combinations. For example, a membrane formed by depositing a layer of the polyurethane of the invention on a porous substrate, can utilize a commercially available membranes having appropriate pore sizes and pore. If a porous substrate having a desired pore size and/or pore density is not commercially available, it is well within the abilities of those of skill in the art to prepare the necessary substrate.

The membranes of the invention are formed by methods known in the art. See, for example, Mizutani, Y. et al., J. Appl. Polym. Sci. 1990, 39, 1087-1100), Breitbach, L. et al., Angew. Makromol. Chem. 1991, 184, 183-196 and Bryjak, M. et al., Angew. Makromol. Chem. 1992, 200, 93-108). The membranes are prepared from the pure polyurethane copolymer, or from mixes of the copolymer and another polymer. The polyurethane membranes of the invention can be laid down on a substrate, e.g., a porous substrate, or they can be prepared without a substrate.

An exemplary membrane of the invention is an ion exchange membrane. The most common functional groups in cation-exchange membranes are sulfonic acid (SO₃H) and carboxylic acid (—COOH). The Nafion brand perfluorosulfonated polymer membranes groups are examples of the first type. See, for example, Meares, P. In Mass Transfer and Kinetics of Ion Exchange; Liberti, L.; Helffefich, F. G., Eds.; NATO ASI Series E: Applied Science No. 71; Martinus Nijhoff Publishers, The Hague, The Netherlands, (1983); pp 329-366; Yeager, H. L. et al., In Perfluorinated Ionomer Membranes; Yeager, H. L.; Eisenberg, Eds.; ACS Symposium Series 180; American Chemical Society: Washington, D.C., (1982); pp 1-6.

The functional groups in anion-exchange membranes are usually quaternary ammonium [—N⁺(CH₃)₃] and to a lesser extent quaternary phosphonium [—P⁺(CH₃) 3] and tertiary sulfonium [—S⁺(CH₃)₂]. Anion-exchange membranes are frequently less stable than cation-exchange membranes because the basic groups are inherently less stable than the acidic groups (Strathmann, H. In Synthetic Membranes: Science, Engineering and Applications; Bungay, P. M.; Lonsdale, H. K.; de Pinho, M. N., Eds.; NATO ASI Series C: Mathematical and Physical Sciences Vol. 181; D. Reidel Publishing Company: Dordrecht, Holland, (1986); pp 1-37).

Other membranes based upon the versatile chemistry of the polyurethanes provided by the invention will be apparent to those of skill in the art. For example, the polyurethane of the invention can also be incorporated into affinity purification membranes in which the affinity for an analyte of a membrane-bound binding functionality is exploited to purify that analyte. Although the materials of the invention can be used in a range of affinity purification protocols, two methodologies are currently preferred. In the first, the porous material is incubated with a fluid containing the analyte. Following the incubation, the membrane is removed from the fluid and the analyte is freed from the membrane. In a second embodiment, the membrane includes a binding functionality that, because of its affinity for the analyte, facilitates the transport of the analyte across the membrane.

The concept of facilitated transport across membranes is recognized in the art. See, for example, Lakshmi et al., Nature 388(21), 758-760 (1997); Noble, Chem. Eng. Progr. 85: 58-70 (1989); Noble et al., J. Membr. Sci. 75: 121-129 (1992). Briefly, the concept of facilitated transport involves the conjugation to a membrane of a species selective for an analyte. The membrane-conjugated species recognizes the analyte and binds to or otherwise forms a complex with the analyte. Thus, the present invention provides materials and methods for achieving the affinity purification of species through a facilitated transport mechanism.

Methods of Using the Devices

The devices of the present invention are useful for the isolation and detection of analytes. In particular, chips of the invention are useful in in performing assays of substantially any format including, but not limited to chromatographic capture, immunoassays, competitive assays, DNA or RNA binding assays, fluorescence in situ hybridization (FISH), protein and nucleic acid profiling assays, sandwich assays and the like. The following discussion focuses on the use of a chip to practice exemplary assays. This focus is for clarity of illustration only and is not intended to define or limit the scope of the invention. Those of skill in the art will appreciate that the method of the invention is broadly applicable to any assay technique for detecting the presence and/or amount of an analyte.

Chips with hydrogels functionalized with energy absorbing moieties are useful in laser desorption mass spectrometry to aid in the desorption and ionization of analytes without further addition of matrix to the chip.

Chromatographic resins of this invention, when functionalized with binding moieties, are useful in the capture and purification of molecules from mixtures.

Membranes of this invention are useful for the isolation of analytes on the membrane surface, followed by their detection.

Detection

The chips of this invention are useful for the detection of analyte molecules. When the hydrogel is functionalized with a binding group, the chip will capture onto the surface analytes that bind to the particular group. Unbound materials can be washed off, and the analyte can be detected in any number of ways including, for example, a gas phase ion spectrometry method, an optical method, an electrochemical method, atomic force microscopy and a radio frequency method. Gas phase ion spectrometry methods are described herein. Of particular interest is the use of mass spectrometry and, in particular, SELDI. Optical methods include, for example, detection of fluorescence, luminescence, chemiluminescence, absorbance, reflectance, transmittance, birefringence or refractive index (e.g., surface plasmon resonance, ellipsometry, quartz crystal microbalance, a resonant mirror method, a grating coupler waveguide method (e.g., wavelength-interrogated optical sensor (“WIOS”) or interferometry). Optical methods include microscopy (both confocal and non-confocal), imaging methods and non-imaging methods. Immunoassays in various formats (e.g., ELISA) are popular methods for detection of analytes captured on a solid phase. Electrochemical methods include voltametry and amperometry methods. Radio frequency methods include multipolar resonance spectroscopy or interferometry. Optical methods include microscopy (both confocal and non-confocal), imaging methods and non-imaging methods. Immunoassays in various formats (e.g., ELISA) are popular methods for detection of analytes captured on a solid phase. Electrochemical methods include voltametry and amperometry methods. Radio frequency methods include multipolar resonance spectroscopy.

Mass Spectroscopy/SEND

Desorption detectors comprise means for desorbing the analyte from the adsorbent and means for directly detecting the desorbed analyte. That is, the desorption detector detects desorbed analyte without an intermediate step of capturing the analyte in another solid phase and subjecting it to subsequent analysis. Detection of an analyte normally will involve detection of signal strength. This, in turn, reflects the quantity of analyte adsorbed to the adsorbent.

The desorption detector also can include other elements, e.g., a means to accelerate the desorbed analyte toward the detector, and a means for determining the time-of-flight of the analyte from desorption to detection by the detector.

A preferred desorption detector is a laser desorption/ionization mass spectrometer, which is well known in the art. The mass spectrometer includes a port into which the substrate that carries the adsorbed analytes, e.g., a probe, is inserted. Striking the analyte with energy, such as laser energy desorbs the analyte. Striking the analyte with the laser results in desorption of the intact analyte into the flight tube and its ionization. The flight tube generally defines a vacuum space. Electrified plates in a portion of the vacuum tube create an electrical potential which accelerate the ionized analyte toward the detector. A clock measures the time of flight and the system electronics determines velocity of the analyte and converts this to mass. As any person skilled in the art understands, any of these elements can be combined with other elements described herein in the assembly of desorption detectors that employ various means of desorption, acceleration, detection, measurement of time, etc. An exemplary detector further includes a means for translating the surface so that any spot on the array is brought into line with the laser beam.

When the method of detection involves a laser desorption/ionization process, hydrogels of this invention that are functionalized with EAMs, and that optionally are further functionalized with a binding functionality, are particularly useful. The analyte is deposited on the hydrogel and then analyzed by the laser desorption process without further application of matrix, as in traditional MALDI.

Fluorescence and Luminescence

For the detection of low concentrations of analytes in the field of diagnostics, the methods of chemiluminescence and electrochemiluminescence are gaining wide spread acceptance. These methods of chemiluminescence and electro-chemiluminescence provide a means to detect low concentrations of analytes by amplifying the number of luminescent molecules or photon generating events many-fold, the resulting “signal amplification” then allowing for detection of low concentration analytes.

In another embodiment, a fluorescent label is used to label one or more assay component or region of the chip. Fluorescent labels have the advantage of requiring few precautions in handling, and being amenable to high-throughput visualization techniques (optical analysis including digitization of the image for analysis in an integrated system comprising a computer). Preferred labels are typically characterized by one or more of the following: high sensitivity, high stability, low background, low environmental sensitivity and high specificity in labeling. Many fluorescent labels are commercially available from the SIGMA chemical company (Saint Louis, Mo.), Molecular Probes (Eugene, Oreg.), R&D systems (Minneapolis, Minn.), Pharmacia LKB Biotechnology (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersburg, Md.), Fluka Chemica-Biochemika Analytika (Fluka Chemie AG, Buchs, Switzerland), and Applied Biosystems (Foster City, Calif.), as well as many other commercial sources known to one of skill. Furthermore, those of skill in the art will recognize how to select an appropriate fluorophore for a particular application and, if it not readily available commercially, will be able to synthesize the necessary fluorophor de novo or synthetically modify commercially available fluorescent compounds to arrive at the desired fluorescent label.

In addition to small molecule fluorophores, naturally occurring fluorescent proteins and engineered analogues of such proteins are useful in the present invention. Such proteins include, for example, green fluorescent proteins of cnidarians (Ward et al., Photochem. Photobiol. 35:803-808 (1982); Levine et al., Comp. Biochem. Physiol., 72B:77-85 (1982)), yellow fluorescent protein from Vibrio fischeri strain (Baldwin et al., Biochemistry 29:5509-15 (1990)), Peridinin-chlorophyll from the dinoflagellate Symbiodinium sp. (Morris et al., Plant Molecular Biology 24:673:77 (1994)), phycobiliproteins from marine cyanobacteria, such as Synechococcus, e.g., phycoerythrin and phycocyanin (Wilbanks et al., J. Biol. Chem. 268:1226-35 (1993)), and the like.

Microscopic Methods

Microscopic techniques of use in practicing the invention include, but are not limited to, simple light microscopy, confocal microscopy, polarized light microscopy, atomic force microscopy (Hu et al., Langmuir 13:5114-5119 (1997)), scanning tunneling microscopy (Evoy et al., J. Vac. Sci. Technol A 15:1438-1441, Part 2 (1997)), and the like.

Spectroscopic Methods

Spectroscopic techniques of use in practicing the present invention include, for example, infrared spectroscopy (Zhao et al., Langmuir 13:2359-2362 (1997)), raman spectroscopy (Zhu et al., Chem. Phys. Lett. 265:334-340 (1997)), X-ray photoelectron spectroscopy (Jiang et al., Bioelectroch. Bioener. 42:15-23 (1997)) and the like. Visible and ultraviolet spectroscopies are also of use in the present invention.

Assays

The chip of the present invention is useful for performing retentate chromatography. Retentate chromatography has many uses in biology and medicine. These uses include combinatorial biochemical separation and purification of analytes, protein profiling of biological samples, the study of differential protein expression and molecular recognition events, diagnostics and drug discovery.

One basic use of retentate chromatography as an analytical tool involves exposing a sample to a combinatorial assortment of different adsorbent/eluant combinations and detecting the behavior of the analyte under the different conditions. This both purifies the analyte and identifies conditions useful for detecting the analyte in a sample. Substrates having adsorbents identified in this way can be used as specific detectors of the analyte or analytes. In a progressive extraction method, a sample is exposed to a first adsorbent/eluant combination and the wash, depleted of analytes that are adsorbed by the first adsorbent, is exposed to a second adsorbent to deplete it of other analytes. Selectivity conditions identified to retain analytes also can be used in preparative purification procedures in which an impure sample containing an analyte is exposed, sequentially, to adsorbents that retain it, impurities are removed, and the retained analyte is collected from the adsorbent for a subsequent round. See, for example, U.S. Pat. No. 6,225,047.

In other applications, chip-based assays based on specific binding reactions are useful to detect a wide variety of targets such as drugs, hormones, enzymes, proteins, antibodies, and infectious agents in various biological fluids and tissue samples. In general, the assays consist of a target, a binding functionality for the target, and a means of detecting the target after its immobilization by the binding functionality (e.g., a detectable label). Immunological assays involve reactions between immunoglobulins (antibodies), which are capable of binding with specific antigenic determinants of various compounds and materials (antigens). Other types of reactions include binding between avidin and biotin, protein A and immunoglobulins, lectins and sugar moieties and the like. See, for example, U.S. Pat. No. 4,313,734, issued to Leuvering; U.S. Pat. No. 4,435,504, issued to Zuk; U.S. Pat. Nos. 4,452,901 and 4,960,691, issued to Gordon; and U.S. Pat. No. 3,893,808, issued to Campbell.

The present invention provides a chip useful for performing assays that are useful for confirming the presence or absence of a target in a sample and for quantitating a target in a sample. An exemplary assay format with which the invention can be used is an immunoassay, e.g., competitive assays, and sandwich assays. Those of skill in the art will appreciate that the invention described herein can be practiced in conjunction with a number of other assay formats.

The chip and method of the present invention are also of use in screening libraries of compounds, such as combinatorial libraries. The synthesis and screening of chemical libraries to identify compounds, which have novel bioactivities, and material science properties is now a common practice. Libraries that have been synthesized include, for example, collections of oligonucleotides, oligopeptides, and small and large molecular weight organic or inorganic molecules. See, Moran et al., PCT Publication WO 97/35198, published Sep. 25, 1997; Baindur et al., PCT Publication WO 96/40732, published Dec. 19, 1996; Gallop et al., J. Med. Chem. 37:1233-51 (1994).

Virtually any type of compound library can be probed using the method of the invention, including peptides, nucleic acids, saccharides, small and large molecular weight organic and inorganic compounds. In a presently preferred embodiment, the libraries synthesized comprise more than 10 unique compounds, preferably more than 100 unique compounds and more preferably more than 1000 unique compounds.

In an exemplary embodiment, a binding domain of a receptor, for example, serves as the focal point for a drug discovery assay, where, for example, the receptor is immobilized, and incubated both with agents (i.e., ligands) known to interact with the binding domain thereof, and a quantity of a particular drug or inhibitory agent under test. The extent to which the drug binds with the receptor and thereby inhibits receptor-ligand complex formation can then be measured. Such possibilities for drug discovery assays are contemplated herein and are considered within the scope of the present invention. Other focal points and appropriate assay formats will be apparent to those of skill in the art.

Analytes

The methods of the present invention can be used to detect any target, or class of targets, which interact with a binding functionality in a detectable manner. The interaction between the target and binding functionality can be any physicochemical interaction, including covalent bonding, ionic bonding, hydrogen bonding, van der Waals interactions, attractive electronic interactions and hydrophobic/hydrophilic interactions.

In a preferred embodiment, the target molecule is a biomolecule such as a polypeptide (e.g., peptide or protein), a polynucleotide (e.g., oligonucleotide or nucleic acid), a carbohydrate (e.g., simple or complex carbohydrate) or a lipid (e.g., fatty acid or polyglycerides, phospholipids, etc.). In the case of proteins, the nature of the target can depend upon the nature of the binding functionality. For example, one can capture a ligand using a receptor for the ligand as a binding functionality; an antigen using an antibody against the antigen, or a substrate using an enzyme that acts on the substrate.

The target can be derived from any sort of biological source, including body fluids such as blood, serum, saliva, urine, seminal fluid, seminal plasma, lymph, and the like. It also includes extracts from biological samples, such as cell lysates, cell culture media, or the like. For example, cell lysate samples are optionally derived from, e.g., primary tissue or cells, cultured tissue or cells, normal tissue or cells, diseased tissue or cells, benign tissue or cells, cancerous tissue or cells, salivary glandular tissue or cells, intestinal tissue or cells, neural tissue or cells, renal tissue or cells, lymphatic tissue or cells, bladder tissue or cells, prostatic tissue or cells, urogenital tissues or cells, tumoral tissue or cells, tumoral neovasculature tissue or cells, or the like.

The target can be labeled with a fluorophore or other detectable group either directly or indirectly through interacting with a second species to which a detectable group is bound. When a second labeled species is used as an indirect labeling agent, it is selected from any species that is known to interact with the target species. Preferred second labeled species include, but are not limited to, antibodies, aptazymes, aptamers, streptavidin, and biotin.

The target can be labeled either before or after it interacts with the binding functionality. The target molecule can be labeled with a detectable group or more than one detectable group. Where the target species is multiply labeled with more than one detectable group, the groups are preferably distinguishable from each other. Properties on the basis of which the individual quantum dots can be distinguished include, but are not limited to, fluorescence wavelength, absorption wavelength, fluorescence emission, fluorescence absorption, ultraviolet light absorbance, visible light absorbance, fluorescence quantum yield, fluorescence lifetime, light scattering and combinations thereof.

Methods of Making

In another exemplary embodiment, the invention provides a method of making a device of the invention. The method includes contacting a substrate with a polyurethane described herein, such that the polyurethane is immobilized on the substrate.

In another embodiment, the invention provides a method for making a plurality of adsorbent devices. Each member of the plurality of devices includes: (a) a solid support having a surface; and (b) an adsorbent polyurethane film reversibly or irreversibly immobilized on the surface. In a preferred method, each solid support is contacted with an aliquot of the polyurethane sampled from a single batch of the polyurethane. The solid-support polyurethane construct is optionally heated, to immobilize the polyurethane on the solid support's surface.

In an exemplary embodiment, the polyurethane is immobilized on the substrate at a plurality of addressable locations.

The use of a single batch of polyurethane minimizes chip-to-chip and lot-to-lot variations. A preferred size for a single batch of the polyurethane is from about 0.5 liters and 5 liters. The single batch is preferably of sufficient volume to prepare a total area of addressable locations of least about 500,000 mm², preferably from about 500,000 mm² to about 50,000,000 mm², more preferably from about 100,000 to about 5,000,000 addressable locations.

After synthesis, the functionalized film components can be further elaborated by a variety of chemical reactions well known to those skilled in the art. For example, in order to produce an anion exchange polyurethane, the reactive polyurethane is mixed with a suitable amine (e.g. dimethylethanol amine or trimethyl amine) and allowed to react to produce a quaternary ion exchange site. Production of an analogous polyurethane, containing cation exchange sites can be accomplished by a number of well-known synthetic schemes. A particularly versatile method relies on the use of a dimethyl sulfide displacement reaction, in which a reactive polyurethane is first reacted with a solution of dimethyl sulfide. The resulting reaction product is a sulfonium based anion exchange polyurethane. A second cation exchange site generation reagent is then added to the reaction mixture, which can be heated in order to help drive the reaction to completion. An exemplary reagent for this purpose is mercaptopropionic acid. A solution of this acid is first pH adjusted to about 11 and then mixed with the above suspension of sulfonium based anion exchange polyurethane. After heating the suspension at about 70° C. for a predetermined period of time, the substitution reaction is complete and the resulting functionalized film component is now a weak acid cation exchange polymer.

Similar reaction pathways are available for preparing polyurethanes with other binding functionalities. It is within the abilities of one of skill in the art to determine an appropriate reaction pathway to conjugate a selected binding functionality to the functionalized film components of use in the chips of the invention (see, for example, Hernanson, BIOCONJUGATE TECHNIQUES, Academic Press, San Diego, 1996; and Dunn et al., Eds. POLYMERIC DRUGS AND DRUG DELIVERY SYSTEMS, ACS Symposium Series Vol. 469, American Chemical Society, Washington, D.C. 1991.

Following the synthesis and functionalization steps set forth above, the functionalized film components are coated onto the solid support, which optionally includes a linker arm that interacts with the polyurethane. Thus, in an exemplary embodiment, a slurry of the polyurethane is aliquoted onto the solid support surface at the location of the previously grafted linker arm. The slurry of particles is allowed to react for a selected period of time and then the residual unattached polyurethane are simply rinsed away.

The following examples are provided to illustrate selected embodiments of the invention and are not to be construed as limiting its scope.

### EXAMPLES

### Example 1

### Non-Functionalized T-Gel

1.1 Preparative Method for a Non-Functionalized Polyurethane Polymer Unit—T-Gel

### 1.1a PU-400 Isocyante-Terminated Polyurethane from PEG 400

Toluene di-isocyanate (“TDI”) (1.15 g) was added in one portion to a mixture of poly(ethyleneglycol) (“PEG”) 400 (1.2 g), and trimethylol propane (“TMP”) (0.134 g) in anhydrous dimethylformamide (13 g). The mixture was stirred for 1 h, forming the T-gel polyurethane polymer.

### 1.1b PU-200 Isocyante-Terminated Polyurethane (T-Gel) from PEG 200

The procedure was the same as above except PEG 200 (0.55 g) was used instead of PEG 400.

### 1.1c PU-600 Isocyante-Terminated Polyurethane (T-Gel) from PEG 600

The procedure was the same as 1.1a above except PEG 600 (1.8 g) was used.

### 1.1d PU-1000 Isocyante-Terminated Polyurethane from PEG 1000

The procedure was the same as 1.1a above except PEG 1000 (2.96 g) was used.

### 1.1e PU-Dihydroxybenzoic Acid (DHBA) Isocyante-Terminated Polyurethane from DHBA

TDI (10.9 g) was added in one portion to a mixture of DHBA (4.71 g), and TMP (1.34 g) in dimethylformamide (236 g). The mixture was stirred for 1 h, forming the T-gel polyurethane polymer.

### Example 2

### T-Gel with Cationic Exchange Functionalities

2.1 Preparation of a Polyurethane T-Gel Weak Cation Exchange Polymer

### 2.1a 1,4-Butanediol-3-carboxylic Acid-Based PU Polymer

The procedures were the same as in Example 1.1a except 1,4-butanediol-3-carboxylic acid (0.41 g) was added instead of PEG 400. The solution was used to prepare WCX chips. Alternatively, some of the 1,4-butanediol-3 carboxylic acid was partially replaced by PEG 200, 400, or 1000.

### 2.1b Glycolic Acid-Based PU Polymer from T-Gel

Glycolic acid (4.4 mg) was added to 5% T-gel (1 g) already prepared from example 1.1b. The solution was used to prepare WCX chips. Alternatively, T-gels from any of Examples 1.1a to 1.1 d can be used.

### 2.2 Preparation of a Polyurethane Strong Cation Exchange Polymer T-Gel

The procedures are the same as Example 2.1a except 1,4-butanediol-3-sulfonic acid (0.56 g) was added instead of PEG 400. The solution was used to prepare SCX chips. Alternatively, some of the 1,4-butanediol-3-sulfonic acid was partially replaced by PEG 200, 400, or 1000.

### Example 3

### T-Gel with Anion Exchange Functionalities

3.1 Preparation of a Polyurethane Strong Anion Exchange Polymer

### 3.1a 1,4-Butanediol-3-trimethylammonium Chloride-Based PU Polymer

The procedure used to prepare the strong anion exchange polymer are the same as Example 2.1a except 1,4-butanediol-3-trimethylammonium chloride (0.55 g) was added instead of 1,4-butanediol-3-carboxylic acid. Alternatively, some of the 1,4-butanediol-3-trimethylammonium chloride was partially replaced by PEG 200, 400, or 1000.

### 3.1b Choline Chloride-Based PU Polymer T-Gel

The preparative method for a choline strong anion exchange polymer was the same as example 2.1b except choline chloride (8 mg) was added to the T-gel instead of glycolic acid. The solution was used to prepare SAX chips. Alternatively, choline chloride can be added to the T-gels from any of examples 1.1a to 1.1d.

### Example 4

### SEND Hydrogels

4.1 Preparation of a SEND EAM-Polyurethane Polymer

### 4.1a α-Cyano-4-hydroxycinamic Acid-Based PU polymer

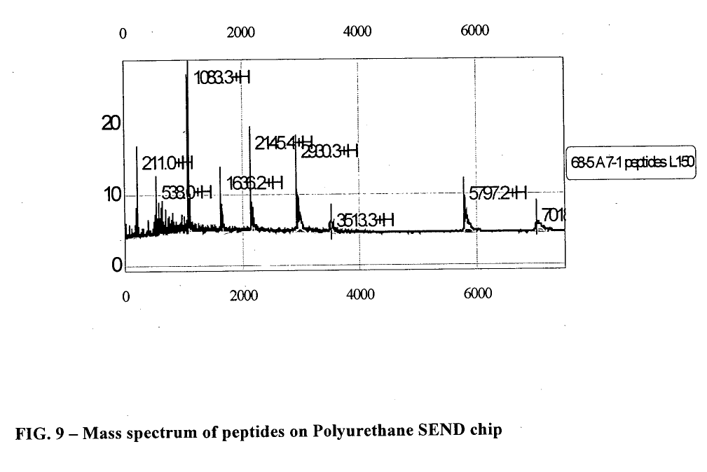

A 2.5% solution of the T-gel from example 1 lb (isocyanate-terminated PU200) and α-cyano-4-hydroxycinamic acid (CHCA) (11 mg) were mixed to form a SEND polyurethane polymer. Alternatively, PU-400, PU600 and PU 1000 T-gels can be used. The solution is ready to prepare CHCA SEND chips. One microliter of this solution was applied to an aluminum substrate coated with silicon dioxide and baked for 2 hours at 80° C. The SEND chip was shown to launch 7 peptide mixtures in SELDI MS without adding any EAM as shown in FIG. 9.

### 4.1b Sinapinic Acid-Based PU Polymer from T-Gel

The procedures are the same as 4.1a except sinapinic acid (13 mg) was used instead of CHCA. The solution was used to prepare SPA SEND chips.

### Example 5

### Hydrogels with Reactive and Adsorbent Functional Groups

5.1 Preparation of Imidazole-Functionalized PU Polymer from T-Gel

The procedure was the same as Example 4.1a, except imidazole (3.9 mg) was used instead of CHCA. The solution was used to prepare imidazole functionalized chips. One microliter of this solution was applied to an aluminum substrate coated with silicon dioxide and baked for 2 hours at 80° C.

5.2 Preparation of Epoxy-Functionalized PU Polymer

The procedure was the same as Example 4.1a, except glycidol (4.3 mg) was used instead of CHCA. The solution was used to prepare epoxy functionalized chips. One microliter of this solution was applied to an aluminum substrate coated with silicon dioxide and baked for 2 hours at 80° C.

5.3 Preparation of Epoxy-Functionalized PU-SEND Polymer

The procedure was the same as Example 4.1a, except glycidol (4.3 mg) was also used along with CHCA. The solution is ready to prepared PU-EPOXY-SEND chips. One microliter of this diluted solution (2.5%) was applied to an aluminum substrate coated with silicon dioxide and baked for 2 hours at 80° C.

5.3 Preparation of N-hydroxysuccinimide-Functionalized PU Polymer

The procedure was the same as Example 4.1a, except N-hydroxysuccinimide (6.6 mg) was used instead of CHCA. The solution was used to prepare N-hydroxysuccinimide chips. One microliter of this solution was applied to an aluminum substrate coated with silicon dioxide and baked for 2 hours at 80° C.

5.4 Preparation of C16-Functionalized Hydrophobic PU Polymer

The procedure was the same as Example 4.1a, except 1-dodecyl alcohol (14 mg) was used instead of CHCA. The solution was used to prepare hydrophobic chips. One microliter of this solution was applied to an aluminum substrate coated with silicon dioxide and baked for 2 hours at 80° C.

5.5 Preparation of a Metal Chelating Agent-Based IMAC PU Polymer

The procedure was the same as Example 4.1a, except N-hydroxyl-ethylethylenediaminetriacetic acid (16 mg) was used instead of CHCA. The solution was used to prepare IMAC chips. Alternatively, T-gel from PU-400 and PU 1000 can be used. One microliter of this solution was applied to an aluminum substrate coated with silicon dioxide and baked for 2 hours at 80° C.

5.6 Preparation of a Heparin-Based PU Polymer

The procedure was the same as Example 4.1a, except sodium salt of heparin (14 mg) was used instead of CHCA. The solution was used to prepare chips. One microliter of this solution was applied to an aluminum substrate coated with silicon dioxide and baked for 2 hours at 80° C. Alternatively, PU-400 and PU 1000 can be used.

5.7 Preparation of Hydrazine PU Polymer

The chips prepared from T-gels from 1.1a to 1.1d were partially cured for 30 min at 80° C. These chips were immersed in 1% hydrazine for 15 min. After being washed and dried, the hydrazine was used to capture glycoproteins by formation of an imine followed by reduction. Alternatively, PU-400 and PU-1000 T-gels can be used.

### Example 6

### Preparation of Chips

6.1 Preparation of Chips Including PU Polymers

One microliter of solution from each of Examples 1-5 was spotted on different locations of one or more aluminum substrates coated with silicon dioxide. Alternatively, the solution was spin-coated onto glass chips. The substrate-polymer construct is cured for 2 hours at 80° C.

All publications and patent documents cited in this application are incorporated by reference in their entirety for all purposes to the same extent as if each individual publication or patent document were so individually denoted. By their citation of various references in this document, Applicants do not admit any particular reference is “prior art” to their invention.

1. A device comprising:
(a) a solid support comprising a surface; and
(b) a polyurethane hydrogel attached to said surface by an interaction which is a member selected from physisorption and chemisorption,
wherein
said hydrogel comprises a plurality of urethane bonds; and
a member selected from a binding functionality, an energy absorbing moiety and combinations thereof.

2. The device according to claim 1, wherein at least two of said urethane bonds are converted to urea bonds by cross-linking with a moiety comprising at least two isocyanate groups.

3. The device of claim 1, wherein said hydrogel comprises a copolymer between at least:
(i) a cross-linking monomer comprising at least three reactive moieties selected from the group consisting of a hydroxyl moiety, a thiol moiety and combinations thereof;
(ii) a first monomer comprising two reactive moieties selected from the group consisting of a hydroxyl moiety, a thiol moiety and combinations thereof;
(iii) a second monomer comprising at least two reactive moieties selected from the group consisting of an isocyanate moiety, an isothiocyanate moiety and combinations thereof; and
(iv) a member selected from a binding functionality monomer, an energy absorbing monomer and combinations thereof.

4. The device according to claim 3, wherein said cross-linking monomer is an alkyl polyol comprising no more than 6 carbon atoms.

5. The device according to claim 3, wherein said first monomer has the formula: 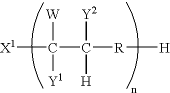
wherein
X¹ is a member selected from OH and SH;
Y¹ and Y² are members independently selected from H, substituted or unsubstituted alkyl, substituted or unsubstituted heteroalkyl, substituted or unsubstituted aryl, substituted or unsubstituted heteroaryl, substituted or unsubstituted heteroarylalkyl, positively charged moieties, negatively charged moieties, metal complexing moieties, metal complexes, hydrophilic moieties, hydrophobic moieties, reactive organic functional groups and combinations thereof;
W is H or a halogen;
R is a member selected from O, S, NH₂ and alkyl substituted with a member selected from O, S and NH₂; and
n is an integer from 1 to 1000.

6. The device according to claim 3, wherein said second monomer has the formula:
Z¹=C═N—R¹—N═C=Z²
wherein
R¹ is a member selected from substituted or unsubstituted alkyl, substituted or unsubstituted heteroalkyl, substituted or unsubstituted aryl, substituted or unsubstituted heteroaryl, and substituted or unsubstituted heterocycloalkyl moieties, and
Z¹ and Z² are independently selected from O and S.

7. The device according to claim 3, wherein said first monomer is a poly(alkylene oxide).

8. The device according to claim 6, wherein R¹ is a member selected from substituted or unsubstituted C₄-C₂₂ alkyl and substituted or unsubstituted C₆-C₁₂ aryl.

9. The device according to claim 3, wherein
(i) said cross-linking moiety is a member selected from trimethylol propane and butanetriol;
(ii) said first monomer is poly(ethylene glycol); and
(ii) said second monomer is a member selected from toluenediisocyanate, cyclohexyldiisocyanate, butyldiisocyanate and hexyldiisocyanate.

10. The device according to claim 1, wherein said binding functionality monomer comprises a binding functionality selected the group consisting of a biospecific moiety, a positively charged moiety, a negatively charged moiety, an anion exchange moiety, a cation exchange moiety, a metal ion complexing moiety, a metal complex, a polar moiety, a hydrophobic moiety and a reactive organic functional group.

11. The device according to claim 1, wherein said binding functionality monomer comprises a binding functionality selected from the group consisting of an amino acid, a dye, a carbohydrate, a nucleic acid, a polypeptide, a lipid and a sugar.

12. The device according to claim 1, wherein said binding functionality monomer comprises a binding functionality selected from an antibody, an antigen, ligands for receptors, receptors, heparin, biotin, avidin, and streptavidin.

13. The device according to claim 1, wherein said binding functionality monomer comprises a metal ion complexing moiety selected from N,N-bis(carboxymethyl)-L-lysine, iminodiacetic acid, aminohydroxamic acid, salicylaldehyde, 8-hydroxy-quinoline, N,N,N′-tris(carboxytrimethyl)ethanolamine, N-(2-pyridylmethyl)aminoacetate.

14. The device according to claim 1, wherein said binding functionality monomer comprises a binding functionality selected from diethylamine, triethylamine, sulfonate and carboxylate.

15. The device according to claim 1, wherein said binding functionality monomer comprises a binding functionality selected from expoxy, imdazole, N-hydroxy-succinimide, iodoacetyl, thiol and aldehyde.

16. The device according to claim 1, wherein said binding functionality comprises a complexed metal ion selected from copper, iron nickel cobalt, gallium and zinc.

17. The device according to claim 1, wherein said EAM monomer comprises an energy absorbing moiety comprising a photo-reactive moiety comprising an aryl nucleus that absorbs photo-irradiation from a source, generating thermal energy, and transferring said thermal energy to promote desorption and ionization of an analyte in operative contact with said hydrogel.

18. The device according to claim 17, wherein said EAM monomer comprises an energy absorbing moiety selected from benzoic acid, cinnamic acid, succinic acid, sinapinic acid, nicotinic acid and derivatives thereof.

19. The device according to claim 1, wherein said solid support is selected from the group consisting of a chip, a chromatographic resin and a membrane.

20. The device according to claim 1, wherein said solid support is coated with silicon dioxide and said polyurethane is covalently immobilized on said surface.

21. The device according to claim 1, wherein said solid support comprises at least one addressable feature having said polyurethane attached thereto.

22. The device according to claim 1, further comprising an analyte interacting with said binding functionality.

23. The device according to claim 22, wherein said interacting is a member selected from the group consisting of covalent bonding, ionic bonding, hydrogen bonding, van der Waals interactions, repulsive electronic interactions, attractive electronic interactions, hydrophobic interactions, hydrophilic interactions.

24. The device according to claim 22, wherein the analyte comprises a member selected from a polypeptide, a nucleic acid and combinations thereof.

25. A method of detecting an analyte, said method comprising:
(a) contacting a sample comprising said analyte with said device of claim 1, thereby adsorbing said analyte on said device; and
(b) detecting the adsorbed analyte.

26. The method according to claim 28, wherein said adsorbed analyte is detected directly on the device, or it is detected after being desorbed from said device.

27. The method according to claim 28, wherein said analyte is detected by mass spectrometry, or a method detecting fluorescence, luminescence, chemiluminescence, absorbance, reflectance, transmittance, birefringence, refractive index.

28. The method according to claim 27, further comprising:
(c) applying an energy absorbing matrix device to said bound analyte; and
(d) detecting said analyte by laser desorption/ionization mass spectrometry.

29. The method according to claim 27, wherein said sample further comprises a contaminant, said method further comprising, prior to said detecting,
(e) washing said contaminant from said device.

30. A method for making a plurality of adsorbent devices, each member of said plurality comprising:
(a) a solid support comprising a surface; and
(b) an adsorbent polyurethane film immobilized on said surface, wherein said polyurethane is a copolymer between at least:
(i) a cross-linking monomer comprising at least three reactive moieties selected from the group consisting of a hydroxyl moiety, a thiol moiety and combinations thereof;
(ii) a first monomer comprising two reactive moieties selected from the group consisting of a hydroxyl moiety, a thiol moiety and combinations thereof;
(iii) a second monomer comprising at least two reactive moieties selected from the group consisting of an isocyanate moiety, an isothiocyanate moiety and combinations thereof; and
(iv) a member selected from a binding functionality monomer, an energy absorbing monomer and combinations thereof;
said method comprising:
(1) contacting each said solid support with an aliquot of said polyurethane, wherein each aliquot is sampled from a single batch of said polyurethane, thereby forming a plurality of polyurethane-coated solid supports; and
(2) curing each polyurethane-coated solid support, thereby immobilizing said polyurethane on said surface and forming said plurality of adsorbent devices.

31. The method of claim 30, wherein the polyurethane is immobilized on each substrate surface at a plurality of addressable locations on the surface.

32. The method of claim 30, wherein said single batch has a volume between 0.5 liters and 5 liters.

33. The method of claim 31, wherein the total area of the addressable locations made from said single batch is at least 500,000 mm².

34. The method of claim 33, wherein the total area of the addressable locations made from said single batch is between 500,000 mm² and 50,000,000 mm².

35. The method of claim 31, wherein said plurality of addressable locations includes between 100,000 and 5,000,000 addressable locations.

36. A kit comprising:
(a) a solid support comprising a surface;
(b) a container comprising a polyurethane functionalized film precursor, said polyurethane is a copolymer between at least:
(i) a cross-linking monomer comprising at least three reactive moieties selected from the group consisting of a hydroxyl moiety, a thiol moiety and combinations thereof;
(ii) a first monomer comprising two reactive moieties selected from the group consisting of a hydroxyl moiety, a thiol moiety and combinations thereof;
(iii) a second monomer comprising at least two reactive moieties selected from the group consisting of an isocyanate moiety, an isothiocyanate moiety and combinations thereof; and
(iv) a member selected from a binding functionality monomer, an energy absorbing monomer and combinations thereof; and
(c) instructions for immobilizing said functionalized film precursor on said surface.

37. The kit according to claim 36, wherein said binding moieties are isocyanates.

38. The kit according to claim 37, further comprising:
(d) directions for chemically converting said isocyanates to a different binding functionality.

39. A polyurethane hydrogel comprising:
(i) a member selected from a binding functionality, an energy absorbing moiety and combinations thereof, and
(ii) cross-linked polyurethane moieties, wherein said polyurethane moieties are a product of a reaction between polyurethane units, each unit comprising a plurality of isocyanate or isothiocyanate moieties and a plurality of internal urethane bonds, wherein links between the units are formed by reaction of isocyanate or isothiocyante moieties with at least one of said plurality of internal urethane bonds.

40. A polyurethane hydrogel unit that is copolymer between at least:
(i) a cross-linking monomer comprising at least three reactive moieties selected from the group consisting of a hydroxyl moiety, a thiol moiety and combinations thereof;
(ii) a first monomer comprising two reactive moieties selected from the group consisting of a hydroxyl moiety, a thiol moiety and combinations thereof;
(iii) a second monomer comprising at least two reactive moieties selected from the group consisting of an isocyanate moiety, an isothiocyanate moiety and combinations thereof, and
(iv) a member selected from a binding functionality monomer, an energy absorbing monomer and combinations thereof.
